SEQUENCE LISTING (208 sequences)

SEQ ID NO: 1 - protein, 98 aa, Homo sapiens
  VARIANT (33)..(33): X may be Ala or Gly
QVQLVQSGAEVKKPGSSVKVSCKASGGTFSSYXISWVRQAPGQGLEWMGXIXXXXGTANYAQKFQGRVTITADESTSTAYMELSSLRSEDTAVYYCAR

SEQ ID NO: 2 - protein, 98 aa, Homo sapiens
  VARIANT (31)..(31): X may be Asp, Asn,  Ser or Thr
EVQLLESGGGLVQPGGSLRLSCAASGFTFSXYXMXWVRQAPGKGLEWVSXIXXXGXSTYYADSVKGRFTISRDNSKNTLYLQMNSLRAEDTAVYYCAK

SEQ ID NO: 3 - protein, 98 aa, Homo sapiens
  VARIANT (31)..(31): X may be Asn, Ser or Thr
EVQLVQSGAEVKKPGESLKISCKGSGYSFTXYWIXWVRQMPGKGLEWMGXIXPXDSXTRYSPSFQGQVTISADKSISTAYLQWSSLKASDTAMYYCAR

SEQ ID NO: 4 - protein, 11 aa, Homo sapiens
WGQGTLVTVSS

SEQ ID NO: 5 - protein, 88 aa, Homo sapiens
  VARIANT (30)..(30): X may be Ala, Asp, Asn, Ser or Arg
DIQMTQSPSSLSASVGDRVTITCRASQSIXXXLNWYQQKPGKAPKLLIYXASSLQSGVPSRFSGSGSGTDFTLTISSLQPEDFATYYC

SEQ ID NO: 6 - protein, 88 aa, Homo sapiens
  VARIANT (30)..(30): X may be Ala, Asn, Asp, Arg, or Ser
EIVLTQSPATLSLSPGERATLSCRASQSVXXXLAWYQQKPGQAPRLLIYXASNRATGIPARFSGSGSGTDFTLTISSLEPEDFAVYYC

SEQ ID NO: 7 - protein, 89 aa, Homo sapiens
  VARIANT (30)..(30): X may be Asn, Asp, Arg, Ser, or Thr
EIVLTQSPATLSLSPGERATLSCRASQSVXXXXLAWYQQKPGQAPRLLIYXASSRATGIPDRFSGSGSGTDFTLTISSLEPEDFAVYYC

SEQ ID NO: 8 - protein, 94 aa, Homo sapiens
  VARIANT (31)..(31): X may be Ala, His, Phe, Ser, or Tyr
DIVMTQSPDSLAVSLGERATINCKSSQSVLXSSNNXNXLAWYQQKPGQPPKLLIYXASTRESGVPDRFSGSGSGTDFTLTISSLQAEDVAVYYC

SEQ ID NO: 9 - protein, 9 aa, Homo sapiens
  VARIANT (3)..(3): X may be Ala, Asp, Arg, Gly, His, Phe, Pro, Ser, or Tyr
QQXXXXPXT

SEQ ID NO: 10 - protein, 10 aa, Homo sapiens
FGQGTKVEIK

SEQ ID NO: 11 - protein, 863 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 12 - protein, 915 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEESSYGSPFTPAKRPKRKVAPKRRQERPVAPPKKRRRKIHRMDHYAAETRQDKMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 13 - protein, 888 aa, Homo sapiens
  misc_feature (599)..(599): Xaa can be any naturally occurring amino acid
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKXRGXRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 14 - protein, 862 aa, Mus spps
MARQGCFGSYQVISLFTFAIGVNLCLGFTASRIKRAEWDEGPPTVLSDSPWTNTSGSCKGRCFELQEVGPPDCRCDNLCKSYSSCCHDFDELCLKTARGWECTKDRCGEVRNEENACHCSEDCLSRGDCCTNYQVVCKGESHWVDDDCEEIRVPECPAGFVRPPLIIFSVDGFRASYMKKGSKVMPNIEKLRSCGTHAPYMRPVYPTKTFPNLYTLATGLYPESHGIVGNSMYDPVFDATFHLRGREKFNHRWWGGQPLWITATKQGVRAGTFFWSVSIPHERRILTILQWLSLPDNERPSVYAFYSEQPDFSGHKYGPFGPEMTNPLREIDKTVGQLMDGLKQLKLHRCVNVIFVGDHGMEDVTCDRTEFLSNYLTNVDDITLVPGTLGRIRPKIPNNLKYDPKAIIANLTCKKPDQHFKPYMKQHLPKRLHYANNRRIEDLHLLVERRWHVARKPLDVYKKPSGKCFFQGDHGFDNKVNSMQTVFVGYGPTFKYRTKVPPFENIELYNVMCDLLGLKPAPNNGTHGSLNHLLRTNTFRPTLPEEVSRPNYPGIMYLQSDFDLGCTCDDKVEPKNKLEELNKRLHTKGSTEERHLLYGRPAVLYRTSYDILYHTDFESGYSEIFLMPLWTSYTISKQAEVSSIPEHLTNCVRPDVRVSPGFSQNCLAYKNDKQMSYGFLFPPYLSSSPEAKYDAFLVTNMVPMYPAFKRVWTYFQRVLVKKYASERNGVNVISGPIFDYNYNGLRDIEDEIKQYVEGSSIPVPTHYYSIITSCLDFTQPADKCDGPLSVSSFILPHRPDNDESCNSSEDESKWVEELMKMHTARVRDIEHLTGLDFYRKTSRSYSEILTLKTYLHTYESEI

SEQ ID NO: 15 - protein, 8 aa, Artificial sequence
  Synthetic polymer for binding metal ions
GSHHHHHH

SEQ ID NO: 16 - protein, 110 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARNWSEALYVVFDY

SEQ ID NO: 17 - protein, 97 aa, Homo sapiens
EIVLTQSPATLSLSPGERATLSCRASQSVANFLAWYQQKPGQAPRLLIYYASNRATGIPARFSGSGSGTDFTLTISSLEPEDFAVYYCQQYFSAPFT

SEQ ID NO: 18 - protein, 108 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGLADFWYFDY

SEQ ID NO: 19 - protein, 97 aa, Homo sapiens
DIQMTQSPSSLSASVGDRVTITCRASQSIDVWLNWYQQKPGKAPKLLIYAASSLQSGVPSRFSGSGSGTDFTLTISSLQPEDFATYYCQQSYSTPLT

SEQ ID NO: 20 - protein, 105 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARPYGTFDY

SEQ ID NO: 21 - protein, 97 aa, Homo sapiens
EIVLTQSPATLSLSPGERATLSCRASQSVADSLAWYQQKPGQAPRLLIYKASNRATGIPARFSGSGSGTDFTLTISSLEPEDFAVYYCQQSNGWPRT

SEQ ID NO: 22 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDDLPLGRLDY

SEQ ID NO: 23 - protein, 97 aa, Homo sapiens
EIVLTQSPATLSLSPGERATLSCRASQSVNKHLAWYQQKPGQAPRLLIYKASNRATGIPARFSGSGSGTDFTLTISSLEPEDFAVYYCQQGIRAPYT

SEQ ID NO: 24 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDLYIYGDLDY

SEQ ID NO: 25 - protein, 98 aa, Homo sapiens
EIVLTQSPATLSLSPGERATLSCRASQSVAKFLAWYQQKPGQAPRLLIYKASNRATGIPARFSGSGSGTDFTLTISSLEPEDFAVYYCQQFNSAPITF

SEQ ID NO: 26 - protein, 110 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPIGTGYYGFFDY

SEQ ID NO: 27 - protein, 97 aa, Homo sapiens
DIQMTQSPSSLSASVGDRVTITCRASQSIGGWLNWYQQKPGKAPKLLIYTASSLQSGVPSRFSGSGSGTDFTLTISSLQPEDFATYYCQQSYTYPIT

SEQ ID NO: 28 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSSAYGDFDY

SEQ ID NO: 29 - protein, 97 aa, Homo sapiens
DIQMTQSPSSLSASVGDRVTITCRASQSIAKWLNWYQQKPGKAPKLLIYFASSLQSGVPSRFSGSGSGTDFTLTISSLQPEDFATYYCQQSYSTPLT

SEQ ID NO: 30 - protein, 112 aa, Homo sapiens
QVQLVQSGAEVKKPGSSVKVSCKASGGTFSSYAISWVRQAPGQGLEWMGGISPYFGNANYAQKFQGRVTITADESTSTAYMELSSLRSEDTAVYYCARYGDFGGNVFTEFDY

SEQ ID NO: 31 - protein, 97 aa, Homo sapiens
DIQMTQSPSSLSASVGDRVTITCRASQSIDGWLNWYQQKPGKAPKLLIYAASSLQSGVPSRFSGSGSGTDFTLTISSLQPEDFATYYCQQSYSTPLT

SEQ ID NO: 32 - protein, 112 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARIARYARGYMHTFDY

SEQ ID NO: 33 - protein, 98 aa, Homo sapiens
EIVLTQSPGTLSLSPGERATLSCRASQSVNNSDLAWYQQKPGQAPRLLIYTASSRATGIPDRFSGSGSGTDFTLTISRLEPEDFAVYYCQQATTVPLT

SEQ ID NO: 34 - protein, 108 aa, Homo sapiens
QVQLVQSGAEVKKPGSSVKVSCKASGGTFSSYGISWVRQAPGQGLEWMGGIIPIFGTANYAQKFQGRVTITADESTSTAYMELSSLRSEDTAVYYCARDDLPLGMLDY

SEQ ID NO: 35 - protein, 97 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQHLSIPIT

SEQ ID NO: 36 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDRYYFTVLDY

SEQ ID NO: 37 - protein, 98 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYTAFPLT

SEQ ID NO: 38 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDFSVYGDFDY

SEQ ID NO: 39 - protein, 98 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQATAFPLT

SEQ ID NO: 40 - protein, 108 aa, Homo sapiens
QVQLVQSGAEVKKPGSSVKVSCKASGGTFSSYAISWVRQAPGQGLEWMGGIIPIFGNTNYAQKFQGRVTITADESTSTAYMELSSLRSEDTAVYYCARDISGYGDLDY

SEQ ID NO: 41 - protein, 97 aa, Homo sapiens
DIQMTQSPSSLSASVGDRVTITCRASQSIAKWLNWYQQKPGKAPKLLIYAASSLQSGVPSRFSGSGSGTDFTLTISSLQPEDFATYYCQQSYSTPLT

SEQ ID NO: 42 - protein, 108 aa, Homo sapiens
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKNAWEGAMLDY

SEQ ID NO: 43 - protein, 97 aa, Homo sapiens
DIQMTQSPSSLSASVGDRVTITCRASQSIHNWLNWYQQKPGKAPKLLIYAASSLQSGVPSRFSGSGSGTDFTLTISSLQPEDFATYYCQQSYSTPLT

SEQ ID NO: 44 - protein, 110 aa, Homo sapiens
EVQLVQSGGGLVQPGGSLRLSCAASGFTFSSYAMSWVRQAPGKGLEWVSVINSDGSSIYYADSVKGRFTISRDNSKNTLYLQMNSLRAEDTAVYYCAKLEAIDYAWGFDY

SEQ ID NO: 45 - protein, 97 aa, Homo sapiens
EIVLTQSPATLSLSPGERATLSCRASQSVADSLAWYQQKPGQAPRLLIYNASNRATGIPARFSGSGSGTDFTLTISSLEPEDFAVYYCQQSFHWPLT

SEQ ID NO: 46 - protein, 108 aa, Homo sapiens
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKDVKGSATLDY

SEQ ID NO: 47 - protein, 97 aa, Homo sapiens
DIQMTQSPSSLSASVGDRVTITCRASQSIANWLNWYQQKPGKAPKLLIYAASSLQSGVPSRFSGSGSGTDFTLTISSLQPEDFATYYCQQSYSTPLT

SEQ ID NO: 48 - protein, 108 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDASTYGDLDY

SEQ ID NO: 49 - protein, 5 aa, Homo sapiens
SYAIS

SEQ ID NO: 50 - protein, 5 aa, Homo sapiens
SYGIS

SEQ ID NO: 51 - protein, 5 aa, Homo sapiens
SYAMS

SEQ ID NO: 52 - protein, 5 aa, Homo sapiens
SYWIG

SEQ ID NO: 53 - protein, 17 aa, Homo sapiens
GIIPIFGTANYAQKFQG

SEQ ID NO: 54 - protein, 17 aa, Homo sapiens
GIIPIFGNTNYAQKFQG

SEQ ID NO: 55 - protein, 17 aa, Homo sapiens
GISPYFGTANYAQKFQG

SEQ ID NO: 56 - protein, 17 aa, Homo sapiens
AISGSGGSTYYADSVKG

SEQ ID NO: 57 - protein, 17 aa, Homo sapiens
VINSDGSSIYYADSVKG

SEQ ID NO: 58 - protein, 17 aa, Homo sapiens
IIYPGDSDTRYSPSFQG

SEQ ID NO: 59 - protein, 17 aa, Homo sapiens
GIIPIFGNANYAQKFQG

SEQ ID NO: 60 - protein, 17 aa, Homo sapiens
GIIPIFGTTNYAQKFQG

SEQ ID NO: 61 - protein, 10 aa, Homo sapiens
DLYIYGDLDY

SEQ ID NO: 62 - protein, 10 aa, Homo sapiens
DSSAYGDFDY

SEQ ID NO: 63 - protein, 10 aa, Homo sapiens
DASTYGDLDY

SEQ ID NO: 64 - protein, 10 aa, Homo sapiens
DISGYGDLDY

SEQ ID NO: 65 - protein, 10 aa, Homo sapiens
DFSVYGDFDY

SEQ ID NO: 66 - protein, 12 aa, Homo sapiens
PIGTGYYGFFDY

SEQ ID NO: 67 - protein, 14 aa, Homo sapiens
YGDFGGNVFTEFDY

SEQ ID NO: 68 - protein, 14 aa, Homo sapiens
IARYARGYMHTFDY

SEQ ID NO: 69 - protein, 10 aa, Homo sapiens
DDLPLGRLDY

SEQ ID NO: 70 - protein, 10 aa, Homo sapiens
DDLPLGMLDY

SEQ ID NO: 71 - protein, 10 aa, Homo sapiens
DRYYFTVLDY

SEQ ID NO: 72 - protein, 11 aa, Homo sapiens
KNAWEGAMLDY

SEQ ID NO: 73 - protein, 11 aa, Homo sapiens
KDVKGSATLDY

SEQ ID NO: 74 - protein, 13 aa, Homo sapiens
KLEAIDYAWGFDY

SEQ ID NO: 75 - protein, 10 aa, Homo sapiens
GLADFWYFDY

SEQ ID NO: 76 - protein, 7 aa, Homo sapiens
PYGTFDY

SEQ ID NO: 77 - protein, 12 aa, Homo sapiens
NWSEALYVVFDY

SEQ ID NO: 78 - protein, 10 aa, Homo sapiens
DDVALGRLDY

SEQ ID NO: 79 - protein, 12 aa, Homo sapiens
SDLNNVSTVLDY

SEQ ID NO: 80 - protein, 12 aa, Homo sapiens
ADANQLSGYLDY

SEQ ID NO: 81 - protein, 10 aa, Homo sapiens
EDVALGRLDY

SEQ ID NO: 82 - protein, 12 aa, Homo sapiens
HDVNSLWAYFDY

SEQ ID NO: 83 - protein, 12 aa, Homo sapiens
GDYNAIYYALDY

SEQ ID NO: 84 - protein, 10 aa, Homo sapiens
DDLALGRLDY

SEQ ID NO: 85 - protein, 14 aa, Homo sapiens
DDDVNSLASYPLDY

SEQ ID NO: 86 - protein, 14 aa, Homo sapiens
DGDVNSVYSSGLDY

SEQ ID NO: 87 - protein, 12 aa, Homo sapiens
GDANALYDYFDY

SEQ ID NO: 88 - protein, 9 aa, Homo sapiens
DDWLSHFDY

SEQ ID NO: 89 - protein, 13 aa, Homo sapiens
RDGNYLHVDHFDY

SEQ ID NO: 90 - protein, 12 aa, Homo sapiens
DHGYAYYSAFDY

SEQ ID NO: 91 - protein, 11 aa, Homo sapiens
DFLSQFSWLDY

SEQ ID NO: 92 - protein, 11 aa, Homo sapiens
DGVTTYDYFDY

SEQ ID NO: 93 - protein, 10 aa, Homo sapiens
HYLPDATLDY

SEQ ID NO: 94 - protein, 10 aa, Homo sapiens
GTFTYDFLDY

SEQ ID NO: 95 - protein, 10 aa, Homo sapiens
GFVNFSRLDY

SEQ ID NO: 96 - protein, 10 aa, Homo sapiens
DWWVTDILDY

SEQ ID NO: 97 - protein, 12 aa, Homo sapiens
GEANEVYPGLDY

SEQ ID NO: 98 - protein, 13 aa, Homo sapiens
DLYGIDIYYALDY

SEQ ID NO: 99 - protein, 10 aa, Homo sapiens
DTFWFTVFDY

SEQ ID NO: 100 - protein, 12 aa, Homo sapiens
GDANSIYPLFDY

SEQ ID NO: 101 - protein, 14 aa, Homo sapiens
GADSNAVRLVGFDY

SEQ ID NO: 102 - protein, 14 aa, Homo sapiens
EDVNYISTYSEFDY

SEQ ID NO: 103 - protein, 12 aa, Homo sapiens
GDDNEVIYHLDY

SEQ ID NO: 104 - protein, 11 aa, Homo sapiens
RASQSIGGWLN

SEQ ID NO: 105 - protein, 11 aa, Homo sapiens
RASQSIDGWLN

SEQ ID NO: 106 - protein, 11 aa, Homo sapiens
RASQSIAKWLN

SEQ ID NO: 107 - protein, 12 aa, Homo sapiens
RASQSVNNSDLA

SEQ ID NO: 108 - protein, 11 aa, Homo sapiens
RASQSISSYLN

SEQ ID NO: 109 - protein, 11 aa, Homo sapiens
RASQSVNKHLA

SEQ ID NO: 110 - protein, 11 aa, Homo sapiens
RASQSVAKFLA

SEQ ID NO: 111 - protein, 12 aa, Homo sapiens
RASQSVSSSYLA

SEQ ID NO: 112 - protein, 11 aa, Homo sapiens
RASQSIHNWLN

SEQ ID NO: 113 - protein, 11 aa, Homo sapiens
RASQSIANWLN

SEQ ID NO: 114 - protein, 11 aa, Homo sapiens
RASQSVADSLA

SEQ ID NO: 115 - protein, 11 aa, Homo sapiens
RASQSIDVWLN

SEQ ID NO: 116 - protein, 11 aa, Homo sapiens
RASQSVANFLA

SEQ ID NO: 117 - protein, 11 aa, Homo sapiens
RASQSIGKVLN

SEQ ID NO: 118 - protein, 11 aa, Homo sapiens
RASQSIGRSLN

SEQ ID NO: 119 - protein, 11 aa, Homo sapiens
RASQSVSKALA

SEQ ID NO: 120 - protein, 11 aa, Homo sapiens
RASQSVSSFLA

SEQ ID NO: 121 - protein, 11 aa, Homo sapiens
RASQSVNSWLA

SEQ ID NO: 122 - protein, 11 aa, Homo sapiens
RASQSVADFLA

SEQ ID NO: 123 - protein, 11 aa, Homo sapiens
RASQSVSDYLA

SEQ ID NO: 124 - protein, 11 aa, Homo sapiens
RASQSVNKFLA

SEQ ID NO: 125 - protein, 11 aa, Homo sapiens
RASQSVRDWLA

SEQ ID NO: 126 - protein, 11 aa, Homo sapiens
RASQSVRKYLA

SEQ ID NO: 127 - protein, 11 aa, Homo sapiens
RASQSVRNFLA

SEQ ID NO: 128 - protein, 11 aa, Homo sapiens
RASQSVSNWLA

SEQ ID NO: 129 - protein, 11 aa, Homo sapiens
RASQSVNNFLA

SEQ ID NO: 130 - protein, 11 aa, Homo sapiens
RASQSVADWLA

SEQ ID NO: 131 - protein, 11 aa, Homo sapiens
RASQSVSSWLA

SEQ ID NO: 132 - protein, 11 aa, Homo sapiens
RASQSVADYLA

SEQ ID NO: 133 - protein, 11 aa, Homo sapiens
RASQSVAKDLA

SEQ ID NO: 134 - protein, 11 aa, Homo sapiens
RASQSVASALA

SEQ ID NO: 135 - protein, 12 aa, Homo sapiens
RASQSVSKSALA

SEQ ID NO: 136 - protein, 12 aa, Homo sapiens
RASQSVAYSSLA

SEQ ID NO: 137 - protein, 12 aa, Homo sapiens
RASQSVRKSGLA

SEQ ID NO: 138 - protein, 12 aa, Homo sapiens
RASQSVAKSDLA

SEQ ID NO: 139 - protein, 7 aa, Homo sapiens
TASSLQS

SEQ ID NO: 140 - protein, 7 aa, Homo sapiens
AASSLQS

SEQ ID NO: 141 - protein, 7 aa, Homo sapiens
FASSLQS

SEQ ID NO: 142 - protein, 7 aa, Homo sapiens
TASSRAT

SEQ ID NO: 143 - protein, 7 aa, Homo sapiens
KASNRAT

SEQ ID NO: 144 - protein, 7 aa, Homo sapiens
GASSRAT

SEQ ID NO: 145 - protein, 7 aa, Homo sapiens
NASNRAT

SEQ ID NO: 146 - protein, 7 aa, Homo sapiens
YASNRAT

SEQ ID NO: 147 - protein, 7 aa, Homo sapiens
GASSLQS

SEQ ID NO: 148 - protein, 7 aa, Homo sapiens
KASSLQS

SEQ ID NO: 149 - protein, 7 aa, Homo sapiens
TASNRAT

SEQ ID NO: 150 - protein, 7 aa, Homo sapiens
DASNRAT

SEQ ID NO: 151 - protein, 7 aa, Homo sapiens
FASNRAT

SEQ ID NO: 152 - protein, 7 aa, Homo sapiens
GASNRAT

SEQ ID NO: 153 - protein, 7 aa, Homo sapiens
NASSRAT

SEQ ID NO: 154 - protein, 7 aa, Homo sapiens
AASSRAT

SEQ ID NO: 155 - protein, 9 aa, Homo sapiens
QQSYTYPIT

SEQ ID NO: 156 - protein, 9 aa, Homo sapiens
QQSYSTPLT

SEQ ID NO: 157 - protein, 9 aa, Homo sapiens
QQATTVPLT

SEQ ID NO: 158 - protein, 9 aa, Homo sapiens
QQHLSIPIT

SEQ ID NO: 159 - protein, 9 aa, Homo sapiens
QQGIRAPYT

SEQ ID NO: 160 - protein, 9 aa, Homo sapiens
QQFNSAPIT

SEQ ID NO: 161 - protein, 9 aa, Homo sapiens
QQYTAFPLT

SEQ ID NO: 162 - protein, 9 aa, Homo sapiens
QQATAFPLT

SEQ ID NO: 163 - protein, 9 aa, Homo sapiens
QQSFHWPLT

SEQ ID NO: 164 - protein, 9 aa, Homo sapiens
QQSNGWPRT

SEQ ID NO: 165 - protein, 9 aa, Homo sapiens
QQYFSAPFT

SEQ ID NO: 166 - protein, 9 aa, Homo sapiens
QQPISTPLT

SEQ ID NO: 167 - protein, 9 aa, Homo sapiens
QQPFDYPFT

SEQ ID NO: 168 - protein, 9 aa, Homo sapiens
QQPISFPYT

SEQ ID NO: 169 - protein, 9 aa, Homo sapiens
QQGHGWPRT

SEQ ID NO: 170 - protein, 9 aa, Homo sapiens
QQYKNAPRT

SEQ ID NO: 171 - protein, 9 aa, Homo sapiens
QQSHRAPWT

SEQ ID NO: 172 - protein, 9 aa, Homo sapiens
QQGHRWPFT

SEQ ID NO: 173 - protein, 9 aa, Homo sapiens
QQGSTAPYT

SEQ ID NO: 174 - protein, 9 aa, Homo sapiens
QQGSDAPLT

SEQ ID NO: 175 - protein, 9 aa, Homo sapiens
QQYNHAPFT

SEQ ID NO: 176 - protein, 9 aa, Homo sapiens
QQGDGWPWT

SEQ ID NO: 177 - protein, 8 aa, Homo sapiens
QQGDTAPI

SEQ ID NO: 178 - protein, 9 aa, Homo sapiens
QQGYRAPIT

SEQ ID NO: 179 - protein, 9 aa, Homo sapiens
QQRYTAPWT

SEQ ID NO: 180 - protein, 9 aa, Homo sapiens
QQFKGAPLT

SEQ ID NO: 181 - protein, 9 aa, Homo sapiens
QQSFRWPLT

SEQ ID NO: 182 - protein, 9 aa, Homo sapiens
QQFKGWPFT

SEQ ID NO: 183 - protein, 9 aa, Homo sapiens
QQYIRWPIT

SEQ ID NO: 184 - protein, 9 aa, Homo sapiens
QQYISAPLT

SEQ ID NO: 185 - protein, 9 aa, Homo sapiens
QQGINWPFT

SEQ ID NO: 186 - protein, 9 aa, Homo sapiens
QQGGNWPWT

SEQ ID NO: 187 - protein, 9 aa, Homo sapiens
QQYSTWPFT

SEQ ID NO: 188 - protein, 9 aa, Homo sapiens
QQSSRDPWT

SEQ ID NO: 189 - protein, 9 aa, Homo sapiens
QQSSGFPLT

SEQ ID NO: 190 - protein, 9 aa, Homo sapiens
QQSSRYPWT

SEQ ID NO: 191 - protein, 9 aa, Homo sapiens
QQYGSSPLT

SEQ ID NO: 192 - protein, 9 aa, Homo sapiens
QQSSRFPWT

SEQ ID NO: 193 - protein, 9 aa, Homo sapiens
QQDSAFPWT

SEQ ID NO: 194 - protein, 9 aa, Homo sapiens
QQSNTNPLT

SEQ ID NO: 195 - protein, 9 aa, Homo sapiens
QQSNKDPLT

SEQ ID NO: 196 - protein, 9 aa, Homo sapiens
QQSNTLPLT

SEQ ID NO: 197 - protein, 9 aa, Homo sapiens
QQSNTPPLT

SEQ ID NO: 198 - protein, 9 aa, Homo sapiens
QQATKPPLT

SEQ ID NO: 199 - protein, 9 aa, Homo sapiens
QQDYGYPLT

SEQ ID NO: 200 - protein, 108 aa, Homo sapiens
  MUTAGEN (52)..(52): May be Ile or Ser
QVQLVQSGAEVKKPGSSVKVSCKASGGTFSSYAISWVRQAPGQGLEWMGGIXPXFGXXNYAQKFQGRVTITADESTSTAYMELSSLRSEDTAVYYCARDLYIYGXXDY

SEQ ID NO: 201 - protein, 97 aa, Homo sapiens
  MUTAGEN (30)..(30): X may be Ala, Asp or Gly
DIQMTQSPSSLSASVGDRVTITCRASQSIXXWLNWYQQKPGKAPKLLIYXASSLQSGVPSRFSGSGSGTDFTLTISSLQPEDFATYYCQQSYXXPXT

SEQ ID NO: 202 - protein, 109 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARWGQGTLVTVSS

SEQ ID NO: 203 - protein, 109 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTLVTVSS

SEQ ID NO: 204 - protein, 109 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARWGQGTLVTVSS

SEQ ID NO: 205 - protein, 108 aa, Homo sapiens
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 206 - protein, 113 aa, Homo sapiens
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGQGTKVEIK

SEQ ID NO: 207 - protein, 107 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGQGTKVEIK

SEQ ID NO: 208 - protein, 107 aa, Homo sapiens [sequence omitted]

(19) United States
(12) Patent Application Publication
(10) Pub. No.: US 2015/0087812 A1
(43) Pub. Date: Mar. 26, 2015

(54) Human Autotaxin Antibodies and Methods of Use

(71) Applicants: Ellen Chi, San Diego, CA (US); Thai Dinh, San Diego, CA (US); Suzanne Edavettal, San Diego, CA (US); Sandra Fenton, San Diego, CA (US); Damon Hamel, San Diego, CA (US); Sheri Moores, Spring House, PA (US); Jose Pardinas, Spring House, PA (US); H. Mimi Zhou, San Diego, CA (US); Barry L. Ziober, Spring House, PA (US)

(72) Inventors: Ellen Chi, San Diego, CA (US); Thai Dinh, San Diego, CA (US); Suzanne Edavettal, San Diego, CA (US); Sandra Fenton, San Diego, CA (US); Damon Hamel, San Diego, CA (US); Sheri Moores, Spring House, PA (US); Jose Pardinas, Spring House, PA (US); H. Mimi Zhou, San Diego, CA (US); Barry L. Ziober, Spring House, PA (US)

(73) Assignee: JANSSEN BIOTECH, INC., Horsham, PA (US)

(21) Appl. No.: 14/381,227

(22) PCT Filed: Mar. 11, 2013
(86) PCT No.: PCT/US13/30235

§ 371 Date: Aug. 27, 2014

Related U.S. Application Data
(60) Provisional application No. 61/611,241, filed on Mar. 15, 2012.

(51) Int. Cl. C07K 16/40

(52) U.S. Cl. CPC C07K 16/40; C07K 2317/92; C07K 2317/565
USPC 530/387.9; 530/388.26

(57) ABSTRACT

Antibodies and compositions capable of binding and, in a particular aspect, inhibiting the lysophosphatidylipase D activity of human autotaxin protein. The antibodies or are useful in detecting human autotaxin protein and treating diseases and disorders associated with unwanted lysophosphatidylipase D and the presence of lysophosphatidic acid, such as cancer and pulmonary diseases.

### FIELD OF THE INVENTION

The present invention relates to a human antibody capable of binding directly to the lysophospholipase, autotaxin, and in one aspect, neutralizing the catalytic activity of the enzyme as evidenced by the lack of production of lysophosphatidic acid (LPA), and methods of use to treat disease states related to unwanted autotaxin catalytic activity.

### BACKGROUND OF THE INVENTION

Autotaxin (ATX), also known as ectonucleotide pyrophosphatases/phosphodiesterases 2 (ENPP2), is a secreted lysophospholipase D (E.C. 3.1.4.39). In mammals, the enzyme produces phospholipid lysophosphatidic acid (LPA) from lysophosphatidylcholine (LPC). It was first discovered as an autocrine motility factor released by a human melanoma A2058 cell line (Stracke et al., J Biol Chem 267: 2524-2529, 1992). Subsequent independent studies identified ATX as the extracellular enzyme responsible for the generation of LPA from LPC (Tokumura et al., J Biol Chem 277:39436-39442, 2002; Umezu-Goto et al., J Cell Biol 158: 227-233, 2002). ATX may also catalyze the conversion of sphingosylphosphorylcholine (lysophosphingomyelin) to sphingosine-1-phosphate (S1P), which is also a modulator of cell motility.

ATX is a secreted 100 KDa glycoprotein comprising two cysteine-rich N-terminal somatomedin B like domains (SMB1 and SMB2), a central phosphodiesterase domain (catalytic domain) and a C-terminal nuclease-like domain. Three isoforms of ATX have been reported in both human and mouse as a result of alternatively spliced autotaxin transcripts. Human isoform alpha lacks exon 21 and has 915 amino acids, and it is identical to the originally discovered ATX from human melanoma A2058 cells; isoform beta lacks exon 12 and 21 and has 863 amino acids; isoform gamma lacks exon 12 and has 889 amino acids. All three isoforms are identical from amino acid residues 1-324 which includes the catalytic (201-214) and substrate binding domains (244-255). The isoforms of ATX are expressed differentially. Based on quantitative PCR studies, ATX expression is limited to peripheral tissues as the beta isoform; the gamma isoform is mainly expressed in the brain in both mouse and human (Giganti, et al., J Biol Chem 283: 7776-7789, 2008). All three recombinant isoforms catalyze LPC conversion to LPA, with the beta isoform having the strongest hydrolytic activity, followed by the gamma isoform, then the alpha isoform, which has the least stability and activity (Giganti, et al., J Biol Chem 283: 7776-7789, 2008).

The roles of ATX, LPA and S1P in disease pathology, especially, cancer have become an area of intense scientific interest. The cancer promoting properties of ATX have been ascribed to the growth-factor-like responses evoked by LPA in the vast majority of tested cell types (Lee et al., J Biol Chem 271 (40): 24408-12, 1996; Hama et al., J Biol Chem 279: 17634-17639, 2004). LPA and other lysophospholipids signal through specific G-protein coupled receptors formerly known as Edg (endothelial differentiation gene) receptors. LPA receptors Edg-2, Edg-4 and Edg-7 are now also termed as LPA₁, LPA₂ and LPA₃ receptor (Ishii et al., Annu. Rev. Biochem. 73 (2004), 321-354). Additional LPA receptors as well as receptors for sphingosine-derived ATX catabolite sphingosine-1-phosphate (S1P) S1P1 to 5, have also been identified. Signaling of these receptors has been linked to important physiological and pathophysiological effects including cell migration, cell survival, proliferation, and neuropathic pain (Choi et al., Ann. Rev. Pharmacol. Toxicol. 50: 157-186, 2010; Tokumura et al., Am. J. Physiol. 267: C204-C210, 1994; Rizza et al., Lab. Invest. 79: 1227-1235, 1999; Inoue et al., Nat. Med. 10: 712-718, 2004). Indeed, overexpression of either ATX or LPA receptors have been shown to promote tumor formation, angiogenesis and metastasis (Liu et al., Cancer Cell 15: 539-550, 2009; Nam et al., Oncogene 19: 241-247, 2000; Taghavi et al. Oncogene 27: 6806-6816; 2008; Yu et al., J. Natl Cancer Inst. 100: 1630-1642, 2008). At the same time, elevated ATX concentrations have been observed in a number of cancerous tissues (Xu et al., Clin. Cancer Res. 1: 1223-1232, 1995; Eder et al., Clin. Cancer Res. 6: 2482-2491, 2000; Xie et al., J Biol Chem 277: 32516-32526, 2002; Yang et al., Am. J. Respir. Cell. Mol. Biol. 21: 216e222, 1999; Yang et al., Clin. Exp. Metastasis 19: 603-608, 2002). These relationships indicate that ATX is an important drug target and reducing the enzymatic activity of ATX could be beneficial for the treatment of cancer.

ATX has an essential requirement for divalent metal cations, and activity can be inhibited in the presence of metal chelators, such as EDTA, phenanthrolines, and L-histidine, albeit at millimolar concentrations (Clair et al., Lipids Health Dis., 4:5, 2005). LPA causes end-product inhibition of ATX activity, which led to the search for other LPA analogs that were capable of inhibiting ATX (van Meeteren et al., J Biol. Chem. 280: 21155-21161, 2005). Analogs differing from LPA structurally in the details of the hydrophobic tail, the linker region or in the head group itself have inhibition constants from 100 nM to 1 μM. One such molecule is α-bromomethylene phosphonate LPA (BrP-LPA), a phosphatase resistant LPA analog that inhibits ATX and antagonizes LPA receptors in the micromolar range (Jiang et al., Chem Med Chem 2: 679-690, 2007). Another synthetic inhibitor is [4-(tetradecanoylamino)benzyl]phosphonic acid (S32826), which inhibits ATX with an IC50 in the 10 nM range (Ferry et al., J Pharmacol Exp Ther 327:809-819, 2008). LPA analogs may, however, agonize or antagonize a varying spectrum of LPA receptors and thus may not block all LPA functions.

Several monoclonal antibodies have been generated by different groups with full length human ATX or partial ATX peptides (Nakamura et al., Clinica Chimica Acta 388:51-58, 2008; Tanaka et al., FEBS Letters 571: 197-204, 2004; US 2010/0120064A1; WO2011/151461A2), however, no ATX function blocking large biologic molecule, e.g. a monoclonal antibody or fragment, have been reported.

### SUMMARY OF THE INVENTION

The present invention provides monoclonal antibodies capable of binding ATX with high affinity and, in one aspect, blocking the ability of the enzyme to catalyze the production of lysophosphatidic acid (LPA) species from lysophosphatidylcholine. An antibody of the invention is useful as a therapeutic to treat conditions arising from unwanted production of LPA species and the sequelae of biological responses related to binding of LPA to one or more of LPA receptors, now known as LPAR1-6 on cells, tissues, or organs in a host subject. In another aspect of the invention, pairs of ATX binding monoclonal antibodies have been identified that can bind ATX concurrently and, which pairs may also comprise the ability to eliminate ATX from tissues in a host or to neutralize the catalytic activity of ATX.

The antibodies were discovered using a human Fab library displayed on the surface of a bacteriophage attached to the coat protein pIX. The human antibody fragments derived from the phage library represent functional antigen binding fragments that can be configured as fully human antibodies or other constructs useful as effective therapeutics and detection reagents for disease associated with unwanted autotaxin level or activity such as in cancer patients.

Amino acid sequences of exemplary ATX binding human monoclonal antibody fragments are provided which can be encoded by nucleic acids for expression in a host cell. One aspect of the invention is an isolated antibody reactive with human ATX protein having the antigen binding ability of a monoclonal antibody comprising an antigen binding domain comprising amino acid sequences as set forth at specified positions of a human antibody variable domain region, FR1-CDR1-FR2-CDR2-FR3, as set forth in SEQ ID NOs: 1-3 and 5-8, or are an isolated CDR or CDR regions which can be used to create a functional binding protein having at least one variable regions and further comprising the identified CDR1, CDR2, or CDR3 regions in a heavy or light chain human framework.

The isolated variable regions identified as ATX binding regions are represented by SEQ ID NO: 16-48; or an antigen binding domain comprising a CDR with amino acid sequences as set forth in SEQ ID NOs: 49-199 alone or at specified positions as set forth in SEQ ID NOs: 1-3 or 5-8 and variants thereof. In a specific embodiment, the human ATX binding antibody comprises a variable domain comprising a sequence selected from any of the possible VH variants of SEQ ID NO: 200 or the VL variants of 201.

In another embodiment of the invention, the monoclonal antibody binding domains used as full length IgG structures, have constant domains derived from human IgG constant domains or specific variants thereof and are used as therapeutic molecules in a pharmaceutical preparation to cause the inactivation of ATX in the body by enhanced elimination or inhibition of the catalytic function of ATX locally or systemically. In another embodiment, the binding domains are configured as antibody fragments for use as a therapeutic molecule capable of binding of ATX. In one aspect of the invention, there is provided a pharmaceutically acceptable formulation, delivery system, or kit or a method of treating conditions related to the activity of ATX comprising one or more of the ATX binding domains of the invention such as but not limited to 16-48 and 49-199, in particular H-CDR3 as represented by SEQ ID NO: 61-103, the L-CDR3 sequence QQSYSTPL (SEQ ID NO: 156) and VH and VL pairs as provided by the sequence variants of SEQ ID NO: 200 and 201, respectively.

### BRIEF DESCRIPTION OF THE SEVERAL VIEWS OF THE DRAWING

FIG. 1A-B shows the germline gene sequences and numbering system used to build the Fab libraries displayed on pIX coat protein, wherein each of the HV domains consist of a variegated FR1-CDR1-FR2-CDR2-FR3 according to SEQ ID NO: 1-3 (1=IGHV1-69, 2=IGHV3-23, and 3=IGHV5-51) followed by a variable length, variegated H-CDR3 region, and a J-region (FR4, SEQ ID NO: 4) (1A); and each of the LV domains consist of a variegated FR1-CDR1-FR2-CDR2-FR3 according to SEQ ID NO: 5-8 (5=IGKV1-39 (012), 6=IGKV3-11 (L6), 7-IGKV3-20 (A27), and 8=IGKV4-1 (B3)) followed by a CDR3 according to SEQ ID NO: 9 which is followed by a J-region (FR4, SEQ ID NO: 10) (1B). IGHV1-69: SEQ ID NO: 202; IGHV3-23: SEQ ID NO: 203; IGHV5-51: SEQ ID NO: 204; IGKV3-20 (A27): SEQ ID NO: 205; IGKV4-1 (B3): SEQ ID NO: 206; IGKV3-11 (L6): SEQ ID NO: 207; IGKV1-39 (012) SEQ ID NO: 208.

FIG. 2 is a column graph of showing the relative inhibition of the artificial substrate, FS-3, hydrolysis by human ATX beta isoform (2.5 nM) for 18 MAbs tested at 2.5 microgram per ml and a human IgG1 control Mab.

FIG. 3 is a graph showing the concentration dependent inhibition of enzymatic conversion of LPC to LPA by human ATX beta isoform (1.5 nM) using the coupled choline oxidation assay by six neutralizing Mabs and the calculated IC50 for each. The compound S32826 is a small molecule ATX end product analog inhibitor.

FIG. 4 is a graph showing the concentration dependent inhibition of enzymatic conversion of LPC to LPA by mouse ATX using the coupled choline oxidation assay by six neutralizing Mabs and the calculated IC50 for each. The compound S32826 is a small molecule ATX end product analog inhibitor.

### DETAILED DESCRIPTION OF THE INVENTION

All publications, including but not limited to patents and patent applications, cited in this specification are herein incorporated by reference as though fully set forth.

### ABBREVIATIONS

ATX=autotaxin; BSA=bovine serum albumin; CDR=complementarity determining region; Fab=fragment antigen-binding; F(ab′)2=structure which is two Fab′ monomers; FR=framework; H=heavy chain; IC50=half maximal inhibitory concentration; Ig=immunoglobulin; L=light chain; LPA=lysophosphatidic acid; LPC=lysophosphocholine; Mab=monoclonal antibody; sphingosylphosphorylcholine=SPC; sphingosine-1-phosphate=S1P; PBS=phosphate buffered saline; VL=Variable light chain; VH=Variable heavy chain

### DEFINITIONS

As used herein, an “antibody” includes whole antibodies and any antigen binding fragment or a single chain thereof. Thus, the antibody includes any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein, which can be incorporated into an antibody of the present invention. The term “antibody” is further intended to encompass antibodies, digestion fragments, specified portions and variants thereof, including antibody mimetics or comprising portions of antibodies that mimic the structure and/or function of an antibody or a specified fragment or portion thereof, including single chain and single domain antibodies and fragments thereof. Functional fragments include antigen-binding fragments to a preselected target. Examples of binding fragments encompassed within the term “antigen binding portion” of an antibody include (i) a Fab fragment, a monovalent fragment consisting of the VL, VH, CL and CH, domains; (ii) a F(ab′)2 fragment, a bivalent fragment comprising two Fab fragments linked by a disulfide bridge at the hinge region; (iii) a Fd fragment consisting of the VH and CH, domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (I 988) Science 242:423-426, and Huston et al. (1988) Proc. Natl. Acad Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Conversely, libraries of scFv constructs can be used to screen for antigen binding capability and then, using conventional techniques, spliced to other DNA encoding human germline gene sequences. One example of such a library is the “HuCAL: Human Combinatorial Antibody Library” (Knappik, A. et al. J Mol Biol (2000) 296(1):57-86).

The term “CDR” refers to the complementarity determining region or hypervariable region amino acid residues of an antibody that participate in or are responsible for antigen-specific binding. The hypervariable regions or CDRs of the human IgG subtype of antibody comprise amino acid residues from residues 24-34 (L-CDR1), 50-56 (L-CDR2) and 89-97 (L-CDR3) in the light chain variable domain and 31-35 (H-CDR1), 50-65 (H-CDR2) and 95-102 (H-CDR3) in the heavy chain variable domain as described by Kabat et al. (1991 Sequences of Proteins of Immunological Interest, 5th Ed. Public Health Service, National Institutes of Health, Bethesda, Md.) and/or those residues from a hypervariable loop (i.e., residues 26-32 (L1), 50-52 (L2) and 91-96 (L3) in the light chain variable domain and 26-32 (H1), 53-55 (H2) or the current H2 Chothia definition of 52-57, and 96-101 (H3) in the heavy chain variable domain as described by (Chothia and Lesk, J. Mol. Biol. 196: 901-917 (1987)). Chothia and Lesk refer to structurally conserved HVs as “canonical structures.” Framework or FR1-4 residues are those variable domain residues other than and bracketing the hypervariable regions. The numbering system of Chothia and Lesk takes into account differences in the number of residues in a loop by showing the expansion at specified residues denoted by the small letter notations, e.g., 30a, 30b, 30c, etc. More recently, a universal numbering system has been developed and widely adopted, international ImMunoGeneTics information System® (IMGT) (LaFranc, et al. 2005. Nucl Acids Res. 33:D593-D597).

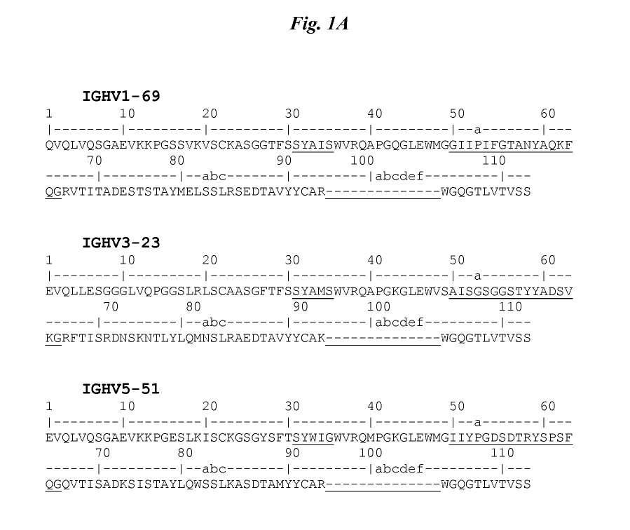

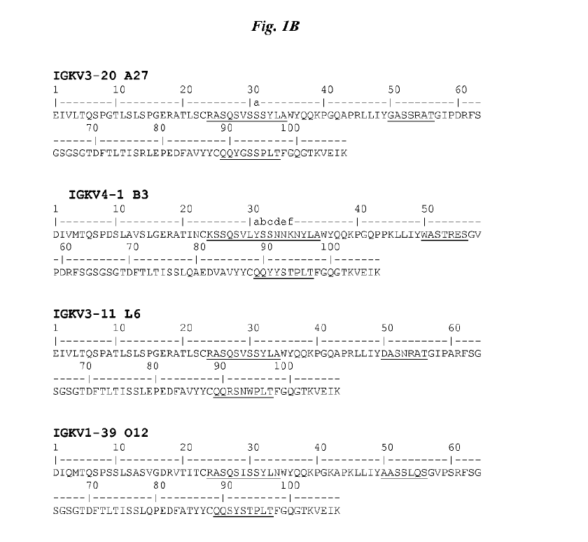

Herein, the CDRs are referred to in terms of both the amino acid sequence and the location within the light or heavy chain by sequential numbering. As the “location” of the CDRs within the structure of the immunoglobulin variable domain is conserved between species and present in structures called loops, by using numbering systems that align variable domain sequences according to structural features, e.g. the numbering system of Kabat; CDR and framework residues and are readily identified. This information is used in grafting and replacement of CDR residues from immunoglobulins of one species into an acceptor framework from, typically, a human antibody (FIG. 1).

The term “maturation” is applied to directed changes in an antibody variable region for the purpose of altering the properties of the polypeptide. As is known in the art and described herein, a large number of positions in the V-region sequences that can impact recognition of antigen. In nature, antibodies achieve high affinity and specificity by the progressive process of somatic mutation. This process can be imitated in vitro to permit parallel selection and targeted variation while maintaining the sequence integrity of each antibody chain such that they reflect the species, in the present case, a human, antibody, while enhancing affinity or a biophysical parameter such as solubility or resistance to oxidation. The process of making directed changes or “maturation” is typically performed at the level of the coding sequence and can be achieved by creating sublibraries for selection of the enhanced property.

As used herein “ATX” refers to an autotaxin polypeptide or a gene or polynucleotide comprising a coding sequence encoding the ATX polypeptide. ATX is also known as ectonucleotide pyrophosphatases/phosphodiesterases 2 (ENPP2), NPP2, ATX-X, PDNP2, LysoPLD, PD-IALPHA. ATX is a secreted lysophospholipase D (E.C. 3.1.4.39) a phospholipase, which catalyzes production of lysophosphatidic acid (LPA) in extracellular fluids but also functions as a phosphodiesterase, which cleaves phosphodiester bonds at the 5′ end of oligonucleotides (EC 3.6.1.9). Human ATX is the product of the human ATX gene (NCBI Gene 5168) located on human chromosome 8q24.1. Three isoforms of ATX have been reported in human from alternatively trans-splicing. Human isoforms including signal peptide and propeptide are given here as alpha (SEQ ID NO: 12, UnitProt Q13822-2, or NCBI# NP006200.3) with 915 amino acids; beta (SEQ ID NO: 11, UnitProt Q13822, or NCBI# NP001035181.1) with 863 amino acids; and gamma (SEQ ID NO: 13, UnitProt Q13822-3, or NCBI# NP001124335.1) with 888 amino acids. The signal and propeptide regions are identical in each isoform where the signal peptide spans residues 1-27 and furin cleavage removes 28-35 (predicted) or 28-48 (Murata et al., JBC 269:48 page 30479), respectively. Isoform alpha has exon 21 deleted and expresses the exon 12 in which a cleavage site is present, leading to a rapid catabolism of the isoform and inactivation of the cleaved protein; Isoform beta has two exons deleted (12 and 21), and isoform gamma has only exon 12 deleted. The ATX sequence is conserved among a number of species. Recombinant mouse ATX beta isoform (UnitProt Q9R1E6, SEQ ID NO: 14) has 94% identity with the human beta isoform over the entire proprotein, 96% in the catalytic domain 201-255), and 100% in the substrate binding region (201-255 of Q13822, SEQ ID NO: 11).

The term “epitope” means a protein determinant capable of specific binding to an antibody. Epitopes usually consist of chemically active surface groupings of molecules, such as amino acids or sugar side chains and usually have specific three-dimensional structural characteristics, as well as specific charge characteristics. Conformational and nonconformational epitopes are distinguished in that the binding to the former but not the latter is lost in the presence of denaturing solvents.

As used herein, K_{D} refers to the dissociation constant, specifically, the antibody K_{D} for a predetermined antigen, and is a measure of affinity of the antibody for a specific target. High affinity antibodies have a K_{D} of 10⁻⁸ M or less, more preferably 10⁻⁹ M or less and even more preferably 10⁻¹⁰ M or less, for a predetermined antigen. The reciprocal of K_{D} is K_{A}, the association constant. The term “k_{dis}” or “k₂,” or “k_{d}” as used herein, is intended to refer to the dissociation rate of a particular antibody-antigen interaction. The “K_{D}” is the ratio of the rate of dissociation (k₂), also called the “off-rate (k_{off})” to the rate of association rate (k₁) or “on-rate (kₒₙ).” Thus, K_{D} equals k₂/k₁ or k_{off}/kₒₙ and is expressed as a molar concentration (M). It follows that the smaller the K_{D}, the stronger the binding. Thus, a K_{D} of 10⁻⁶M (or 1 μM) indicates weak binding compared to 10⁻⁹M (or 1 nM).

As used herein, LPA, indicates any of the lysophosphatidic acids derived from a corresponding lysophosphoroylcholine (LPC). LPC is produced from phosphatidylcholine by the action of an intracellular or extracellular phospholipase (for example, phospholipase A1 or phospholipase A2) hydrolyzing the acyl chain from either position of the glycerol moiety. As the fatty acyl chain and attachment site (sn-1 or sn-2) to the glycerol backbone may vary, there are a number of possible species. LPA molecular species with different acyl chain lengths and saturation are naturally occurring, including 1-palmitoyl (16:0), 1-palmitoleoyl (16:1), 1-stearoyl (18:0), 1-oleoyl (18:1), 1-linoleoyl (18:2), and 1-arachidonyl (20:4) LPA. In addition, infrequently occurring LPA, such as minor alkyl LPA has biological activities similar to acyl LPA.

The terms “monoclonal antibody” or “monoclonal antibody composition” as used herein refer to a preparation of antibody molecules of single molecular composition. A monoclonal antibody composition displays a single binding specificity and affinity for a particular epitope. The term also includes “recombinant antibody” and “recombinant monoclonal antibody” as all antibodies are prepared, expressed, created or isolated by recombinant means, such as (a) antibodies isolated from an animal or a hybridoma prepared by the fusion of antibody secreting animal cells and an fusion partner, (b) antibodies isolated from a host cell transformed to express the antibody, e.g., from a transfectoma, (c) antibodies isolated from a recombinant, combinatorial human or other species antibody library, and (d) antibodies prepared, expressed, created or isolated by any other means that involve splicing of immunoglobulin gene sequences to other DNA sequences. An “isolated antibody,” as used herein, is intended to refer to an antibody which is substantially free of other antibodies having different antigenic specificities. An isolated antibody that specifically binds to an epitope, isoform or variant of human ATX may, however, have cross-reactivity to other related antigens, e.g., from other species (e.g., ATX species homologs). Moreover, an isolated antibody may be substantially free of other cellular material and/or chemicals. In one embodiment of the invention, a combination of “isolated” monoclonal antibodies having different specificities are combined in a well defined composition.

As used herein, “specific binding,” “immunospecific binding” and “binds immunospecifically” refers to antibody binding to a predetermined antigen. Typically, the antibody binds with a dissociation constant (K_{D}) of 10⁻⁷M or less, and binds to the predetermined antigen with a K_{D} that is at least twofold less than its K_{D} for binding to a non-specific antigen (e.g., BSA, casein, or any other specified polypeptide) other than the predetermined antigen. The phrases “an antibody recognizing an antigen” and “an antibody specific for an antigen” are used interchangeably herein with the term “an antibody which binds specifically to an antigen.” As used herein “highly specific” binding means that the relative K_{D} of the antibody for the specific target epitope is at least 10-fold less than the K_{D} for binding that antibody to other ligands.

As used herein, “type” refers to the antibody class (e.g., IgA, IgE, IgM or IgG) that is encoded by heavy chain constant region genes. Some antibody classes further encompass subclasses or “isotypes” which are also encoded by the heavy chain constant regions (e.g., IgG1, IgG2, IgG3 and IgG4). Antibodies may be further decorated by oligosaccharides linked to the protein at specific residues within the constant region domains which further enhance biological functions of the antibody. For example, in human antibody isotypes IgG1, IgG3 and to a lesser extent, IgG2, display effector functions as do murine IgG2a antibodies.

By “effector” functions or “effector positive” is meant that the antibody comprises domains distinct from the antigen specific binding domains capable of interacting with receptors or other blood components such as complement, leading to, for example, the recruitment of macrophages and events leading to destruction of cells bound by the antigen binding domains of the antibody. Antibodies have several effector functions mediated by binding of effector molecules. For example, binding of the C1 component of complement to antibodies activates the complement system. Activation of complement is important in the opsonisation and lysis of cell pathogens. The activation of complement stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

The term “polypeptide” means a molecule that comprises amino acid residues linked by a peptide bond to form a polypeptide. Small polypeptides of less than 50 amino acids may be referred to as “peptides.” Polypeptides may also be referred as “proteins.”

### Overview

The present invention provides isolated Mabs capable of binding and neutralizing the biological activity of mammalian ATX, especially human ATX, and other ATX molecules having the activity of producing LPA from LCA and where the active site includes the consensus sequence as represented by residues 201 to 244 of SEQ ID NO: 11-13 having a Tyr at position 210.

Therefore, the present invention is directed toward the identification of human derived ATX-binding Mabs capable of binding to enzymatically active ATX, and optionally, inhibiting downstream biologic activity resulting from LPA production and biological activity resulting from LPA binding to LPAR, such as LPAR1-6.

### 1. Composition of an Antibody of the Invention

An ATX-binding antibody of the invention is an antibody that binds enzymatically active forms of ATX and, optionally, inhibits, blocks, or interferes with at least one ATX activity or ATX catalytic end product binding, in vitro, in situ and/or in vivo and does not promote, stimulate, induce, or agonize ATX activity. A suitable ATX-binding antibody, specified portion, or variant can also, optionally, affect at least one ATX activity or end product function, such as but not limited to; RNA, DNA or protein synthesis; protein release; cell activation, proliferation or differentiation; antibody secretion; LPA-receptor signaling; ATX cleavage; ATX binding, ATX induction, synthesis or secretion.

The present invention is based upon the discovery of anti-human ATX monoclonal antibodies capable of binding human and mouse ATX. Antibody binding domains in the form of a Fab library displayed on filamentous phage particles linked to the pIX coat protein (see WO29085462A1 and further described herein below) were selected for the ability to bind ATX. Once converted to full Mabs, a competition assay identified those paired VL-VH that, when bound to ATX, prevented other ATX binding Mabs from binding ATX. Alternatively, specified ATX-binding Mabs block the catalytic activity of ATX, such as the conversion of lysophosphatidylcholine to lysophosphatidic acid (LPA) and choline. The ATX-binding antibodies described herein recognize at least two distinct regions on the active form of human ATX protein, indicating the additional discovery of multiple sites on ATX suitable for the ATX binding of antibodies or other compounds with similar function blocking capabilities. Thus, expression and purification of the antibody binding domains provided herein as amino acid sequences further provides the means for selection of novel molecules exhibiting ATX-neutralizing activity.

One embodiment of the invention is an isolated monoclonal antibody that specifically binds to isolated, catalytically active, human autotaxin (ATX) protein with a K_{D} of less than 5×10⁻⁸ M as measured by surface plasmon resonance (SPR).

In some embodiments described herein, the anti-human ATX antibody reduces lysophospholipase D activity of ATX as measured by the conversion of lysophosphatidylcholine to lysophosphatidic acid and choline.

In some embodiments described herein, the anti-human ATX antibody has a binding region comprising a light chain variable (VL) or heavy chain variable (VH) region comprising the amino acid sequence as shown in SEQ ID NO: 16-48 or 200 or 201; and which antibody or binding portion thereof immuno specifically binds ATX. In some embodiments described herein, the anti-human ATX antibody comprises a heavy chain comprising variants as specified in SEQ ID NO: 200 or an antigen binding portion thereof, binds to human ATX protein and, additionally, has the specified functional properties of antibodies of the invention, such as:

binds human ATX with a K_{D} of less than 10 nM,
inhibits the conversion of LPC to LPA and choline in the presence of 2.5 nM human ATX when present at 2.5 ug/ml by at least 20% of the level in the presence of a non-specific IgG 1,
inhibits the conversion of LPC to LPA with an IC50 of less than 1.0 μM, and
is unable to bind to ATX when one of Mabs B6, B8, B13 or B14 is bound to ATX or binds ATX when one of Mabs B10, B15, or B16 is bound to ATX.

In some embodiments described herein, the structural features of the antibodies exhibiting some or all of the above referenced biological activity as described herein and, in particular, the Mabs designated as B6, B8, B13, B14 and B18 binding domains, are used to create structurally related human anti-ATX antibodies that retain at least one functional property of the antibodies of the invention, such as binding to ATX with a K_{D} of less than 10 nM (less than 10⁻⁸ M). More specifically, one or more CDR regions of B6, B8, B13, B14 and B18 (SEQ ID NO: 49, 53, 54, 61-65 and 104-106, 139, 140, and 155 or 156) can be combined recombinantly with known human framework regions and to create additional, recombinantly-engineered, human anti-ATX antibodies of the invention.

In some embodiments described herein, the anti-ATX antibody comprises a heavy chain complementarity determining region (HCDR) 1 (HCDR1) of amino acid residues 35-49 of SEQ ID NO: 200; a HCDR2 of amino acid residues 64-80 of SEQ ID NO: 200; a HCDR3 of amino acid residues 113-122 of SEQ ID NO: 200; a light chain complementarity determining-region (LCDR) 1 (LCDR1) of amino acid residues 24-34 of SEQ ID NO: 201; a LCDR2 of amino acid residues 50-56 of SEQ ID NO: 201; and a LCDR3 of amino acid residues 89-97 of SEQ ID NO: 201.

In some embodiments described herein, the anti-ATX antibody comprises the HCDR1 sequences of SEQ ID NO: 49, the HCDR2 sequences of the sequences selected from the group consisting of SEQ ID NOs: 53 and 54, the HCDR3 sequences selected from the group consisting of SEQ ID NOs: 61-65 and 66, the LCDR1 sequences selected from the group consisting of SEQ ID NOs: 104, 105 and 106, the LCDR2 sequences of the sequences selected from the group consisting of SEQ ID NOs: 139, 140 and 141, and the LCDR3 sequences selected from the group consisting of SEQ ID NOs: 155 and 156.

In some embodiments described herein, the anti-ATX antibody comprises a variable heavy (VH) and a variable light (VL) domain, wherein the VH is derived from IGHV1-69 (SEQ ID NO: 202), IGHV3-23 (SEQ ID NO: 203) or IGHV5-51 (SEQ ID NO: 204) human germline genes and the VL is derived from IGKV1-39 (SEQ ID NO: 208), IGKV3-11 (SEQ ID NO: 207) or IGKV3-20 (SEQ ID NO: 205) human germline genes.

In some embodiments described herein, the anti-ATX antibody VH is derived from IGHV1-69 (SEQ ID NO: 202) human germline gene and the VL is derived from IGKV1-39 (SEQ ID NO: 208) or IGKV3-20 (SEQ ID NO: 205) human germline gene.

In some embodiments described herein, the anti-ATX antibody comprises a consensus sequence for an ATX neutralizing human IGHV1-69 germline derived VH and can be represented as SEQ ID NO: 200: QVQLVQSGAEVKKPGSSVKVSCKASGGTFSSYAISWVRQAPGQGLEWMG GI X₁P X₂FG X₃ X₄NYAQKFQGRVTITADESTSTAYMELSSLRSEDTAVYYCAR DLYIYG X₅ X₆DY wherein X₁ is I or S, X₂ is I or Y, X₃ is N or T, X₄ is A or T, X₅ is D or F and X₅ is F or L.

In some embodiments described herein, the anti-ATX antibody comprises a consensus sequence for an ATX neutralizing human IGVK1-39 germline derived VL and can be represented as SEQ ID NO: 201: DIQMTQSPSSLSASVGDRVTITCRASQSI X₁ X₂WLNWYQQKPGKAPKLLIY X₃ASSLQSGVPSRFSGSGSGTDFTLTISSLQPEDFATYYCQQSYX₄ X₅P X₆T wherein X₁ is A, D or G; X₂ is G or K; and X₃ is A or T; and X₄ is S or T; X₅ is T or Y; and X₆ is I or L.

In one embodiment, the antibodies of the invention have the sequences, including FR1, 2, and/or 3; of IGVH1-69 (SEQ ID NO: 1); IGHV3-23 (SEQ IN NO: 2); or of IGVH5-51 (SEQ ID NO: 3); wherein one or more residues from CDRs selected from the group consisting of SEQ ID NO: 49-199 are present in the CDR position of SEQ ID NO: 1-3, while still retaining the ability of the antibody to bind ATX (e.g., conservative substitutions). Accordingly, in another embodiment, the engineered antibody may be composed of one or more CDRs that are, for example, 90%, 95%, 98% or 99.5% identical to the H-CDRs listed in SEQ ID NOs: 49-103 or of L-CDR3 as given by SEQ ID NO: 155-199.

In addition to simply binding ATX, engineered antibodies, such as those described herein, may be selected for their retention of other functional properties of antibodies of the invention, such as the ability to inhibit ATX protein activity or and prevent formation of catalytic end products thereof, such as LPA, to LPAR positive cells, which binding would result in activation or proliferation of the LPAR positive cells in vivo.

Human monoclonal antibodies of the invention can be tested for binding to ATX by, for example, standard ELISA. Alternatively, human ATX-binding Mabs of the invention can be selected for the inability to bind ATX when one of Mab B6, B8, B13, B14 and B18 providing a neutralizing Mab. Human ATX-binding Mabs that do not neutralize ATX enzymatic activity can be selected by the capacity to bind to ATX when one of Mab B6, B8, B13, B14 and B18 is bound to ATX or by the inability to bind ATX when one of Mab B10, B15, or B16 is bound to ATX.

### 2. Generation of ATX-Neutralizing Antibodies

An ATX-neutralizing antibody exhibiting the desired bioactivity spectrum as exemplified herein by Mabs B1-B18 comprising the heavy chain and light chain sequences can be generated by a variety of techniques.

In another embodiment, the epitope bound by the antibodies of the invention, comprising as few as five to all of residues 201-214 and 244-255 which are believed to be the substrate binding and catalytic residues of ATX beta or all of the mature polypeptide residues 36-863 (SEQ ID NO: 11) or a nucleic acid coding sequence therefore, can be used to immunize a subject in order to produce the antibodies of the invention directly. Such polypeptide or DNA vaccines can also be administered for the purpose of treating, preventing, or ameliorating disease or symptoms of disease associated with the ATX activity.

In one embodiment and as exemplified herein, the human antibody is selected from a phage library, where that phage comprises human immunoglobulin genes and the library expresses human antibody binding domains as, for example, single chain antibodies (scFv), as Fabs, or some other construct exhibiting paired or unpaired antibody variable regions (Vaughan et lo al. Nature Biotechnology 14:309-314 (1996): Sheets et al. PITAS (USA) 95:6157-6162 (1998)); Hoogenboom and Winter, J. Mol. Biol., 227:381 (1991); Marks et al. J. Mol. Biol., 222:581 (1991)). Human monoclonal antibodies of the invention can also be prepared using phage display methods for screening libraries of human immunoglobulin genes. Such phage display methods for isolating human antibodies are established in the art. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Phage clones are selected by and identified through a multi-step procedure known as biopanning. Biopanning is carried out by incubating phage displaying protein ligand variants (a phage display library) with a target, removing unbound phage by a washing technique, and specifically eluting the bound phage. The eluted phage are optionally amplified before being taken through additional cycles of binding and optional amplification that enriches the pool of specific sequences in favor of those phage clones bearing antibody fragments that display the best binding to the target. After several rounds, individual phage clones are characterized, and the sequences of the peptides displayed by the clones are determined by sequencing the corresponding DNA of the phage virion.

### Fab Phage-pIX Library

In a specific embodiment of the phage display technology, a synthetic Fab library displayed on the pIX phage coat protein, described in Shi et al. J Mol Biol 397:385-396, 2010; WO29085462A1 and U.S. Ser. No. 12/546,850 and to be further detailed herein, is used to select binder from a repertoire of human IgG sequences derived from human germline genes. Libraries were constructed on four VL and three VH domains encoded by known IGV and IGJ germline sequences selected based on the frequency which the sequences have been observed to be present in human antibodies isolated from natural sources. The VH, IMGT nomenclature, selected are IGHV1-69 (SEQ ID NO: 1), IGHV3-23 (SEQ ID NO: 2), or IGHV5-51 (SEQ ID NO: 3). The diversity in the VH design produces heavy chains with variable length sequence in the CDR3 region with limited diversity positions in the H-CDR1 and H-CDR2 which remain at a constant length. Framework four (H-FR4) is held constant among all members of the library (FGQGTKVEIK, SEQ ID NO: 4).

VH169, IGHV1-69*01 Length = 98, CDR1 = 31-35,
CDR2 = 50-66
(SEQ ID NO: 1)
QVQLVQSGAE VKKPGSSVKV SCKASGGTFS SYX₁ISWVRQA
PGQGLEWMGX₂ IX₃X₄X₅X₆GTANY AQKFQGRVTI TADESTSTAY
MELSSLRSED TAVYYCAR

Where, in the 169 library X₁ is A or G, X₂ is G or W, X₃ may be I or S, X₄ may be P or A, X₅ may be I or Y and X₆ may be F or N.

VH323, IGHV3-23*01 Length = 98, CDR1 = 31-35,
CDR2 = 50-66
(SEQ ID NO: 2)
EVQLLESGGG LVQPGGSLRL SCAASGFTFS X₁YX₂MX₃WVRQA
PGKGLEWVSX₄ IX₅X₆X₇GX₈STYY ADSVKGRFTI SRDNSKNTLY
LQMNSLRAED TAVYYCAK

Where, in the 323 library, X₁ may be S, D, N, or T; X₂ may be A, G, or W; X₃ may be S or H; X₄ may by V, A, N or G; X₅ may be S, N, K or W; X₆ may be Y, S, G, or Q; X₇ may be S or D; and X₈ may be S or G.

VH551, IGHV5-51*03 Length = 98, CDR1 = 31-35,
CDR2 = 50-66
(SEQ ID NO: 3)
EVQLVQSGAE VKKPGESLKI SCKGSGYSFT X₁YWIX₂WVRQM
PGKGLEWMGX₃ IX₄PX₅DSX₆TRY SPSFQGQVTI SADKSISTAY
LQWSSLKASD TAMYYCAR

Where in the 551 library, X₁ may be S, N, or T; X₂ may be S or G; X₃ may be I or R; X₄ may by D or Y; X₅ may be G or S; X₆ may be D or Y.

A FR4 or JH region having (11 residues), WGQGTLVTVSS (SEQ ID NO: 4), has been joined to the above sequences to form a complete heavy chain variable region.

The H-CDR1 and H-CDR2 positions that were targeted for diversification were determined by 1) diversity in germline genes; and 2) frequency found in contact with antigen in antibody-antigen complexes of known structure (Almagro J Mol Recognit. 17:132-143, 2004). The amino acid diversity at the selected positions was determined by 1) usage in germline; 2) amino acids that are most frequently observed in human rearranged V genes; 3) amino acids predicted to be result from single base somatic mutations; and 4) biochemical and biophysical properties of amino acids that contribute to antigen recognition.

The library incorporates diversity in the CDR3 of the VH (H3) mimicking the repertoire of human antibodies (Shi et al. 2010 supra) as shown below (FORMULA I) where the final length is between 7 and 14 residues. Among the CDR3 of over 5000 human variable regions, amino acids glycine (G) and alanine (A) are frequently used in all positions. In addition, aspartic acid (D) is frequently used in position 95 and tyrosine (Y) is frequently encoded in the positions preceding the canonical region of the J segment Amino acids phenylalanine (F), aspartic acid (D) and tyrosine (Y) predominate at positions 99-101 used in IgGs at these positions. Since these positions often serve as structural support to H-CDR3 and are less accessible to antigen and/or to surface of IgG, amino acids phenylalanine plus leucine (50/50 ratio) at position 99, aspartic acid at position 100 and tyrosine at position 101 were fixed. Thus, the sequence of Formula I is inserted between SEQ ID NOS: 1, 2, or 3 and SEQ ID NO: 4 to create a complete VH.

-(D)-(N)n(N+O)m(F)DY— (I)

### Where:

(D)=Asp (D) and Gly (G) rich position.
(N)n=Ala (A) and Gly (G) rich position, n=3-7.
(O)m=Ala (A), Gly (G) and Y (Tyr) rich in, m=1-4.
(F)=The Phe (F) dominant position.

Various versions of the library encompass pairings with fixed or diversified light chains derived also from the human germline repertoire. In the present invention, the four light-chain library VLₖₐₚₚₐ genes (Kawasaki et al. 2001. Eur J Immunol 31: 1017-1028 and after Schaeble & Zachau, 1993 Biol Chem Hoppe Seyler 374: 1001-1022) are A27 (IGKV3-20*01), B3 (IGKV4-1*01), L6 (IGKV3-11*01), and O12 (IGKV1-39*01) where the gene name in parentheses are the presumed corresponding IMGT gene. The Fabs are displayed on pIX via expression of a dicistronic vector wherein the VH-CH1 domain is fused to the coat protein sequence and the VL-CLkappa or VL-CLlambda is expressed as a free polypeptide which self-associates with the VH-CH1. The CDR regions are underlined.

### Light Chain Variable Library Based on Vkappa (Vk) Germline Genes

O12; IGKV1-39*01, IGKV1D-39*01 Length = 88;
CDR1 = 24-34, CDR2 = 50-56
(SEQ ID NO: 5)
DIQMTQSPSS LSASVGDRVT ITCRASQSI X₁X₂X₃LNWYQQKP
GKAPKLLIYX₄ ASSLQSGVPS RFSGSGSGTD FTLTISSLQP
EDFATYYC
L6, IGKV3-11*01 Length = 88, CDR1 = 24-34,
CDR2 = 50-56
(SEQ ID NO: 6)
EIVLTQSPAT LSLSPGERAT LSCRASQSV X₁X₂X₃LAWYQQKP
GQAPRLLIYX₄ ASNRATGIPA RFSGSGSGTD FTLTISSLEP
EDFAVYYC
A27, IGKV3-20*01, Length = 89, CDR1 = 24-35,
CDR2 = 51-57
(SEQ ID NO: 7)
EIVLTQSPGT LSLSPGERAT LSCRASQSVX₁ X₂X₃X₄LAWYQQK
PGQAPRLLIY X5ASSRATGIP DRFSGSGSGT DFTLTISRLE
PEDFAVYYC
B3, DPK24, VKIVKlobeck; IGKV4-1*01 Length = 94,
CDR1 = 24-40, CDR2 = 56-62
(SEQ ID NO: 8)
DIVMTQSPDS LAVSLGERAT INCKSSQSVL X₁SSNNX₂NX₃LA
WYQQKPGQPP KLLIYX₄ASTR ESGVPDRFSG SGSGTDFTLT
ISSLQAEDVA VYYC

The diversity at the specified positions for each variable region scaffold are summarized in Table 1 below where the amino acids single-letter code is used and is present in the alternative at the specified positions as shown in FIG. 1.

TABLE 1

| | Kabat | O12 | | L6 | | A27 | | B3 | |
|---|---|---|---|---|---|---|---|---|---|
| CDR | Position | SEQ ID NO: 5 | | SEQ ID NO: 6 | | SEQ ID NO: 7 | | SEQ ID NO: 8 | |
| 1 | 30 | X₁ | SRNAD | X₁ | SRNAD | X₁ | SRNTD | | L |
| | 30a | | — | | — | X₂ | SNR | X₁ | YSHFA |
| | 30e | | — | | — | | — | | |
| | 30f | | | | | | | X₂ | KTNE |
| | 31 | X₂ | SNKDG | X₂ | NSKD | X₃ | SNRADH | | N |
| | 32 | X₃ | YHNDW | X₃ | YWDF | X₄ | YFHQSEK | X₃ | YFHNW |
| | | | FSAV | | HSAN | | | | DAS |
| 2 | 50 | X₄ | FYTNKA | X₄ | ADKGY | X₅ | ADGS | X₄ | WSRDY |
| | | | DG | | FTN | | | | A |

The VL CDR3 in all of the libraries has seven residues wherein the first two residues are glutamine (Gln, Q) and the residue corresponding to Kabat residue 95 is proline (Pro, P). For L-CDR3 the sequence corresponds to QQX₁X₂X₃X₄PX₅T (SEQ ID NO: 9), where variegation are as in the table below and at the residue positions are according to Kabat.

TABLE 2

| | Kabat | | | | |
|---|---|---|---|---|---|
| Res- | CDR3 | | | | |
| idue | Position | O12 | L6 | A27 | B3 |
| X1 | 91 | SAYHPD | RYSGF | YSHA | YSHA |
| X2 | 92 | FIYHNDKGRE | RHNSL | YNDSHIFKG | YNDSHIFKG |
| X3 | 93 | STHNDRG | NDKR | SNTDGHR | SNTDGHR |
| X4 | 94 | TYLVFSRGPI | WA | TYLVFAS | TYLVFAS |
| X5 | 96 | LWRFYIN | WYFLIR | WYFLIR | WYFLIR |

As the variable sequence varies in length from gene to gene, diversity in a particular residue location within a hypervariable loop or CDR can be described as follows using the residue numbering as defined in Al-Lazikani B, Lesk A M, Chothia C, 1997 (Standard conformations for the canonical structures of immunoglobulins. J Mol Biol 273: 927-948). In this system, the changes in length of the hypervariable loops are accommodated by the designation of subpositions a, b, c, etc. for a given residue.

A framework 4 (FR4) segment such as JK4, FGQGTKVEIK (SEQ ID NO: 10) was used to form a complete human light chain variable region.

Fab affinity for diverse protein targets from 0.2 to 20 nM has been demonstrated in initial selections.

Methods for an integrated maturation process for improving binding parameters consisting of reshuffling VL or VH diversity or, alternatively, directed or limited VL modification are accomplished using the vectors and primers designed and used for the libraries as described in the referenced publication, as taught herein, and combined with what it known in the art.

### Alternative Sources of ATX-Binding Immunoglobulin Domains

ATX binding antibodies with the characteristics of the human Mabs disclosed herein may be made or binding fragments sourced from immunoglobulin domains formed by a number of methods, including the standard somatic cell hybridization technique (hybridoma method) of Kohler and Milstein (1975) Nature 256:495. In the hybridoma method, a mouse or other appropriate host animal, such as a hamster or macaque monkey, is immunized as described herein to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

An ATX-neutralizing antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-ATX antibody can be isolated from such animals and immortalized using suitable methods, such as the methods described herein. Alternatively, the antibody coding sequences may be cloned, introduced into a suitable vector, and used to transfect a host cell for expression and isolation of the antibody by methods taught herein and those known in the art.

The use of transgenic mice carrying human immunoglobulin (Ig) loci in their germline configuration provide for the isolation of high affinity fully human monoclonal antibodies directed against a variety of targets including human self antigens for which the normal human immune system is tolerant (Lonberg, N. et al., U.S. Pat. No. 5,569,825, U.S. Pat. No. 6,300,129 and 1994, Nature 368:856-9; Green, L. et al., 1994, Nature Genet. 7:13-21; Green, L. & Jakobovits, 1998, Exp. Med. 188:483-95; Lonberg, N and Huszar, D., 1995, Int. Rev. Immunol. 13:65-93; Kucherlapati, et al. U.S. Pat. No. 6,713,610; Bruggemann, M. et al., 1991, Eur. J. Immunol. 21:1323-1326; Fishwild, D. et al., 1996, Nat. Biotechnol. 14:845-851; Mendez, M. et al., 1997, Nat. Genet. 15:146-156; Green, L., 1999, J. Immunol. Methods 231:11-23; Yang, X. et al., 1999, Cancer Res. 59:1236-1243; Brüggemann, M. and Taussig, M J., Curr. Opin. Biotechnol. 8:455-458, 1997; Tomizuka et al. WO02043478). The endogenous immunoglobulin loci in such mice can be disrupted or deleted to eliminate the capacity of the animal to produce antibodies encoded by endogenous genes. In addition, companies, such as Abgenix, Inc. (Freemont, Calif.) and Medarex (San Jose, Calif.) can be engaged to provide human antibodies directed against a selected antigen using technology as described above.

Preparation of polypeptides for use as target ligands in panning strategies and as immunogenic antigens can be performed using any suitable technique, such as recombinant protein production. The target ligand or fragment thereof in the form of purified protein, or protein mixtures including whole cells or cell or tissue extracts, or, in the case of an immunization, the antigen can be formed de novo in the animal's body from nucleic acids encoding said antigen or a portion thereof.

The isolated nucleic acids of the present invention can be made using (a) recombinant methods, (b) synthetic techniques, (c) purification techniques, or combinations thereof, as well-known in the art. DNA encoding the monoclonal antibodies is readily isolated and sequenced using methods known in the art (e.g., by using oligonucleotide probes that are capable of binding specifically to genes encoding the heavy and light chains of human antibody regions). Where a hybridoma is produced, such cells can serve as a source of such DNA. Alternatively, using display techniques wherein the coding sequence and the translation product are linked, such as phage or ribosomal display libraries, the selection of the binder and the nucleic acid is simplified. After phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria.

### Human Antibodies

The invention specifically provides human immunoglobulins (or antibodies) which bind human ATX. These antibodies can also be characterized as engineered or adapted. The immunoglobulins have variable region(s) substantially from a human germline immunoglobulin and include directed variations in residues known to participate in antigen recognition, e.g. the CDRs of Kabat or the hypervariable loops as structurally defined. The constant region(s), if present, are also substantially from a human immunoglobulin. The human antibodies exhibit K_{D} for ATX of at least about 10⁻⁶ M (1 μM), about 10⁻⁷ M (100 nM), 10⁻⁸ (10 nM), 10⁻⁹ M (1 nM), or less than 100 pM (10⁻¹⁰). To affect a change in affinity, e.g., improve affinity or reduce K_{D}, of the human antibody for ATX, substitutions in either the CDR residues or other residues may be made.

The source for production of human antibody which binds to ATX is preferably the sequences provide herein as the variable regions, frameworks and/or CDRs, noted as SEQ ID NO: 16-201 identified as capable of binding human ATX and cross-reacting with ATX homologs of other species, e.g. mouse, using a repertoire of human derived Fab displayed on filamentous phage particles.

The substitution of any of the CDRs into any human variable domain framework is most likely to result in retention of their correct spatial orientation if the human variable domain framework adopts the same or similar conformation to the parent variable framework from which the CDRs originated. The heavy and light chain variable framework regions to be paired in the final Mab can be derived from the same or different human antibody sequences. The human antibody sequences can be the sequences of naturally occurring human antibodies, be derived from human germline immunoglobulin sequences, or can be consensus sequences of several human antibody and/or germline sequences.

Suitable human antibody sequences are identified by computer comparisons of the amino acid sequences of the mouse variable regions with the sequences of known human antibodies. The comparison is performed separately for heavy and light chains but the principles are similar for each.

With regard to the empirical method, it has been found to be particularly convenient to create a library of variant sequences that can be screened for the desired activity, binding affinity or specificity. One format for creation of such a library of variants is a phage display vector.

Another method of determining whether further substitutions are required, and the selection of amino acid residues for substitution, can be accomplished using computer modeling. Computer hardware and software for producing three-dimensional images of immunoglobulin molecules are widely available. In general, molecular models are produced starting from solved structures for immunoglobulin chains or domains thereof. The chains to be modeled are compared for amino acid sequence similarity with chains or domains of solved three dimensional structures, and the chains or domains showing the greatest sequence similarity is/are selected as starting points for construction of the molecular model. The solved starting structures are modified to allow for differences between the actual amino acids in the immunoglobulin chains or domains being modeled, and those in the starting structure. The modified structures are then assembled into a composite immunoglobulin. Finally, the model is refined by energy minimization and by verifying that all atoms are within appropriate distances from one another and that bond lengths and angles are within chemically acceptable limits.

Because of the degeneracy of the code, a variety of nucleic acid sequences will encode each immunoglobulin amino acid sequence. The desired nucleic acid sequences can be produced by de nova solid-phase DNA synthesis or by PCR mutagenesis of an earlier prepared variant of the desired polynucleotide. All nucleic acids encoding the antibodies described in this application are expressly included in the invention.

The variable segments of human antibodies produced as described herein are typically linked to at least a portion of a human immunoglobulin constant region. The antibody will contain both light chain and heavy chain constant regions. The heavy chain constant region usually includes CH1, hinge, CH2, CH3, and, sometimes, CH4 domains.

The human antibodies may comprise any type of constant domains from any class of antibody, including IgM, IgG, IgD, IgA and IgE, and any subclass (isotype), including IgG1, IgG2, IgG3 and IgG4. When it is desired that the humanized antibody exhibit cytotoxic activity, the constant domain is usually a complement-fixing constant domain and the class is typically IgG₁. When such cytotoxic activity is not desirable, the constant domain may be of the IgG₂ class. The humanized antibody may comprise sequences from more than one class or isotype.

Nucleic acids encoding human or humanized light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Such control sequences include a signal sequence, a promoter, an enhancer, and a transcription termination sequence (see Queen et al., Proc. Natl. Acad. Sci. USA 86, 10029 (1989); WO 90/07861; Co et al., J. Immunol. 148, 1149 (1992), which are incorporated herein by reference in their entirety for all purposes).

### 3. Methods of Using an Anti-ATX Antibody

As described in detail below, the present invention demonstrates that isolated monoclonal antibodies having the variable domains of the 57 Fabs listed in Table 3 bind different or overlapping epitopes on ATX and display in vitro and/or in vivo ATX inhibiting activities. Significantly, the reactivity of the selected MAbs includes the ability to dose-dependently block ATX lysophospholipase activity thereby reducing LPA generation in the region surrounding ATX. Thus, the Mabs of the invention can be used to prevent or reduce the effects of LPA binding to local LPA receptors, LPA receptor signal transduction and ameliorate effects caused by LPA-driven cell migration.

Given the properties of the monoclonal antibodies as described in the present invention, the antibodies or antigen binding fragments thereof are suitable both as detecting (diagnostic or prognostic), therapeutic and prophylactic agents for treating or preventing ATX-associated conditions in humans and animals.

In general, use will comprise administering a therapeutically or prophylactically effective amount of one or more monoclonal antibodies or antigen binding fragments of the present invention, or an antibody or molecule selected to have similar spectra of binding and biologic activity, to a susceptible subject or one exhibiting a condition in which ATX activity is known to have pathological sequelae, such as inflammatory or autoimmune disorders or tumor growth and metastasis. Any active form of the antibody can be administered, including Fab and F(ab′)2 fragments.

Preferably, the antibodies used are compatible with the recipient species such that the immune response to the MAbs does not result in an unacceptably short circulating half-life or induce an immune response to the MAbs in the subject. The MAbs administered may exhibit some secondary functions, such as binding to Fc receptors of the subject and activation of ADCC mechanisms, in order to deplete the ATX-associated cell population using cytolytic or cytotoxic mechanisms or they may be engineered to by limited or devoid of these secondary effector functions in order to preserve any ATX-associated cell population.

Treatment of individuals may comprise the administration of a therapeutically effective amount of the antibodies of the present invention. The antibodies can be provided in a kit as described below. The antibodies can be used or administered as a mixture, for example, in equal amounts, or individually, provided in sequence, or administered all at once. In providing a patient with antibodies, or fragments thereof, capable of binding to ATX, or an antibody capable of protecting against ATX in a recipient patient, the dosage of administered agent will vary depending upon such factors as the patient's age, weight, height, sex, general medical condition, previous medical history, etc.

In a similar approach, another therapeutic use of the monoclonal antibodies of the present invention is the active immunization of a patient using an anti-idiotypic antibody raised against one of the present monoclonal antibodies Immunization with an anti-idiotype which mimics the structure of the epitope could elicit an active anti-ATX response (Linthicum, D. S. and Farid, N. R., Anti-idiotypes, Receptors, and Molecular Mimicry (1988), pp 1-5 and 285-300).

The antibodies capable of protecting against unwanted ATX bioactivity are intended to be provided to recipient subjects in an amount sufficient to effect a reduction, resolution, or amelioration in the ATX-related symptom or pathology. An amount is said to be sufficient or a “therapeutically effective amount” to “effect” the reduction of symptoms if the dosage, route of administration, etc. of the agent are sufficient to influence such a response. Responses to antibody administration can be measured by analysis of subject's affected tissues, organs, or cells as by imaging techniques or by ex vivo analysis of tissue samples. An agent is physiologically significant if its presence results in a detectable change in the physiology of a recipient patient.

### Therapeutic Applications

The ATX-neutralizing antibodies of the present invention, antigen binding fragments, or specified variants thereof can be used to measure or cause effects in an cell, tissue, organ or animal (including mammals and humans), to diagnose, monitor, modulate, treat, alleviate, help prevent the incidence of, or reduce the symptoms of, a condition mediated, affected or modulated by ATX or cells expressing ATX. Thus, the present invention provides a method for detecting the presence or concentration of ATX, or modulating or treating at least one ATX related disease, in a cell, tissue, organ, animal, or patient, as known in the art or as described herein, using at least one ATX antibody of the present invention.

ATX is known to be up-regulated in a variety of disease states that involve the release of inflammatory mediators and has been implicated in diverse biological roles including metastatic spread of cancerous cells, proliferation of tumor cells, fibrosis, pain, acute lung injury, sepsis, and inflammation. Particular indications are discussed below. Experimental evidence (WO2011/151461A2) indicated that the level of ATX that is produced by synovial fibroblasts in RA individuals is regulated by tumor necrosis factor alpha (TNF). Addition of TNF to synovial fibroblast derived from healthy individuals led to increased expression of ATX. Moreover, inhibition of TNF in cultures of synovial fibroblasts harvested from an affected joint in an RA patient led to a reduced level of ATX being produced.

### Indications

The biological outcome of ATX action depends on the local availability of its substrates, the presence of regulatory cofactors and the spectrum of LPA receptors expressed on nearby target cells. The major physiological substrate for ATX is LPC. LPC is abundantly present in plasma and serum (at >100 μM) in a predominantly albumin-bound form (The Kₘ of ATX for LPC is approx. 100 which is in the range of LPC plasma concentrations. Blocking ATX enzymatic activity or removal of ATX protein by Mab which specifically bind ATX with a K_{D} of less than 1 μM can be a prophylactic or therapeutic strategy for the treatment of a variety diseases in which ATX production is enhanced and/or deleterious cell responses are produced by binding of the enzymatic products of ATX on lysophospholipids including, but not limited to LPC and sphingosylphosphorylcholine, the downstream effects of the interaction of those products, such as LPA and S1P to cell receptors. Thus, ATX regulates cell activation by changing signaling induced by LPC versus LPA. LPC and ATX may increase locally during inflammation.

LPA₁ (Edg-2), a receptor for LPA is found ubiquitously inhuman tissues: brain, heart, placenta, colon, small intestine, prostate, testis, ovary, pancreas, spleen, kidney, skeletal muscle, and thymus. The nervous system is a major site for lpa₁ expression and which is restricted during embryonic development to the neocortical neurogenic region called ventricular zone, which disappears at the end of cortical neurogenesis, just before birth. Postnatal lpa₁ expression is apparent in and around developing white matter tracts and during the process of myelination by oligodendrocytes as well as in Schwann cells, the myelinating cells of the peripheral nervous system. These observations identify important functions for receptor-mediated LPA signaling in neurogenesis, cell survival, and myelination. Expression of lpa₂ (edg-4) is observed in the testis, kidney, lung, thymus, spleen, and stomach of adult mice and in the human testis, pancreas, prostate, thymus, spleen, and peripheral blood leukocytes. Expression of lpa₂ is upregulated in various cancer cell lines. The LPA₂ receptor may have overlapping specificity and distribution with LPA₁ providing redundancy in the signaling pathways with include MAPK activation, PLC activation, Ca²⁺ mobilization, PI3K/Akt activation and stress fiber formation. The LPA₃ receptor (Edg-7) is distinct from LPA₁ and LPA₂ in its ability to couple G-proteins and is much less responsive to LPA species with saturated acyl chains. Nonetheless, LPA₃ can mediate pleiotropic LPA-induced signaling that includes PLC activation, Ca²⁺ mobilization, AC inhibition/activation, and MAPK activation. LPA₃. Overexpression of LPA₃ in neuroblastoma cells leads, surprisingly, to neurite elongation, whereas that of LPA₁ or LPA₂ results in neurite retraction and cell rounding when stimulated with LPA. Expression of lpa₃ is observed in adult mouse testis, kidney, lung, small intestine, heart, thymus, and brain. In humans, it is found in the heart, pancreas, prostate, testis, lung, ovary, and brain (frontal cortex, hippocampus, and amygdala). LPA₄ (p2y₉/GPR23) is of divergent sequence compared to LPA₁-LPA₃ and more similar to the receptor for another lipid mediator, platelet aggregation factor (PAF). LPA₄ mediates LPA-induced Ca²⁺ mobilization and cAMP accumulation. The lpa₄ gene is expressed at very high levels in the ovary and, to a much lesser extent, in the pancreas, thymus, and human kidney and skeletal muscle (See Ishii et al. 2004 Annual Rev Biochemistry 73: 321-354, for a review).

According to the present invention there is therefore provided the use of an antibody or antibody fragment, which competes for binding to catalytically active ATX with an antibody as described herein. In one aspect, the present invention provides the use of an antibody or antibody fragment selected from the group B1-B18 and related or re-engineered antibodies or fragments thereof in the manufacture of a medicament for the treatment or prophylaxis types and stages of inflammation driven pathology, proliferative and metastatic disease such as cancer, and neurological disorders.

In particular, highly metastatic cancers or cancer progression mechanism which involve angiogenesis wherein one or more cell types secrete ATX or express one or more LPA receptors can be treated with ATX-binding Mabs of the invention. Specific examples of cancers that can be treated by the methods encompassed by the invention include, but are not limited to, Hodgkin lymphoma, follicular lymphoma, glioblastoma, non-small-cell lung cancer, renal cell carcinoma, hepatocellular carcinoma, breast cancer, ovarian cancer, and thyroid carcinomas.

Cancers that can be treated by the methods encompassed by the invention include, but are not limited to, neoplasms, tumors, metastases, or any disease or disorder characterized by uncontrolled cell growth. The cancer may be a primary or metastatic cancer. In a preferred embodiment, the cancer that is being managed, treated or ameliorated in accordance with the methods of the invention is a cancer secreting ATX or one comprised of cells expressing a LPA receptor or where LPA receptor is present on cells in the tumor microenvironment. Additional cancers include, but are not limited to, the following: leukemias such as but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia Vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullaryplasmacytoma; Waldenstrom's macroglobulinemia; bone cancer and connective tissue sarcomas such as but not limited to bone sarcoma, myeloma bone disease, osteosarcoma, chondrosarcoma, Ewing's sarcoma, Paget's disease of bone, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease), and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipidus; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; esophageal cancers such as but not limited to, squamous cancer, adenocarcinoma, adenoid cyctic carcinoma, mucoopidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma, gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, lelomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell cancer, adenocarcinoma, hypernephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (see Vincent T. DeVita, Samuel Hellman, Steven A. Rosenberg. “Cancer: Principles and Practice of Oncology”, (Philadelphia, Pa.: J Lippincott Williams & Wilkins, 2005 7th Ed.). Pre-malignant conditions may also be treated by the methods and compositions of the invention.

Recent reports have shown that ATX is elevated in intra-articular matrix from tissue explants. ATX may be upregulated in adipocytes and adipose tissue ATX is up-regulated in patients exhibiting both insulin resistance and impaired glucose tolerance. These effects may be driven by other inflammatory cytokines such as TNFalpha, IL8, or IL6. Autotaxin released from adipocytes, catalyzes LPA synthesis, and activates preadipocyte proliferation, adipocyte differentiation and obesity. Inhibition of ATX may help control insulin resistance in Type 2 diabetes and obesity. Reduction of LPA may reduce the amount of LPA in oxidized LDL and atherosclerotic plaques thus preventing arterial damage or neointimal proliferation and blockages. Various indications related to effects on cells having one of more LPA receptor type include vascular disease due to inflammatory arthropathy, rheumatoid arthritis, diabetes and related pathologies, obesity, the effects of oxidative damage, the effects of reperfusion after an ischemic event, gastrointestinal tissue inflammation or necrosis, cardiovascular disorders, rejection of tissue transplantation, wound healing, and Alzheimer's disease.

According to the present invention there is therefore provided the use of an antibody or antibody fragment, which competes for binding to catalytically active ATX with and antibody described herein selected from the group B1-B18, in the manufacture of a medicament for the treatment or prophylaxis of a pro-inflammatory process in which ATX directly or indirectly, such as through the release of inflammatory cytokines, leads to pathogenesis in tissues or organs especially in the skin, lungs, and joints. Such pathologies include inflammation related to osteoarthritis, rheumatoid arthritis, psoriatic arthritis, ankylosing spondylitis, neuropathic arthropathy, rheumatic fever, Reiter's syndrome, progressive systemic sclerosis, primary biliary cirrhosis, pemphigus, pemphigoid, necrotizing vasculitis, myasthenia gravis, multiple sclerosis, lupus erythematosus, polymyositis, sarcoidosis, granulomatosis, vasculitis, pernicious anemia, CNS inflammatory disorder, autoimmune haemolytic anemia, Hashimoto's thyroiditis, Graves disease, Reynard's syndrome, glomerulonephritis, dermatomyositis, chronic active hepatitis, celiac disease, autoimmune complications of AIDS, atrophic gastritis, and Addison's disease, endotoxemia or septic shock (sepsis), or one or more of the symptoms of sepsis and other types of acute and chronic inflammation. Those patients who are more particularly able to benefit from the method of the invention are those suffering from infection by E. coli, Haemophilus influenza B, Neisseria meningitides, staphylococci, or pneumococci.

Other conditions that are associated with ATX and amenable to treatment or preventative therapy with the antibodies of the invention include fibrotic disease such as pulmonary fibrosis, diabetic nephropathy, idiopathic pulmonary fibrosis, systemic sclerosis, and cirrhosis.

### Administration and Dosing

The invention provides for stable formulations of an ATX-neutralizing antibody, which is preferably an aqueous phosphate buffered saline or mixed salt solution, as well as preserved solutions and formulations as well as multi-use preserved formulations suitable for pharmaceutical or veterinary use, comprising at least one ATX-neutralizing antibody in a pharmaceutically acceptable formulation. Suitable vehicles and their formulation, inclusive of other human proteins, e.g., human serum albumin, are described, for example, in e.g. Remington: The Science and Practice of Pharmacy, 21st Edition, Troy, D. B. ed., Lipincott Williams and Wilkins, Philadelphia, Pa. 2006, Part 5, Pharmaceutical Manufacturing pp 691-1092, See especially pp. 958-989.

The ATX-binding and/or an ATX-neutralizing antibody in either the stable or preserved formulations or solutions described herein, can be administered to a patient in accordance with the present invention via a variety of delivery methods including intravenous (I.V.); intramuscular (I.M.); subcutaneously (S.C.); transdermal; pulmonary; transmucosal; using a formulation in an implant, pump, cartridge, micropump; or other means appreciated by the skilled artisan, as well-known in the art.

For example, site specific administration may be to body compartment or cavity such as intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, vaginal, rectal, buccal, sublingual, intranasal, or transdermal means.

In general, if administering a systemic dose of the antibody, it is desirable to provide the recipient with a dosage of antibody which is in the range of from about 1 ng/kg-100 ng/kg, 100 ng/kg-500 ng/kg, 500 ng/kg-1 μg/kg, 1 μg/kg-100 μg/kg, 100 μg/kg-500 μg/kg, 500 μg/kg-1 mg/kg, 1 mg/kg-50 mg/kg, 50 mg/kg-100 mg/kg, 100 mg/kg-500 mg/kg (body weight of recipient), although a lower or higher dosage may be administered. Of course, suitable dosages of an antagonist of the present invention will vary, depending upon factors such as the disease or disorder to be treated, the route of administration and the age and weight of the individual to be treated and the nature of the antagonist. Without being bound by any particular dosages, it is believed that for instance for parenteral administration, a daily dosage of from 0.01 to 20 mg/kg of an antibody (or other large molecule) of the present invention (usually present as part of a pharmaceutical composition as indicated above) may be suitable for treating a typical adult.

The treatment may be given in a single dose schedule, or preferably a multiple dose schedule in which a primary course of treatment may be with 1-10 separate doses, followed by other doses given at subsequent time intervals required to maintain and or reinforce the response, for example, at 1-4 months for a second dose, and if needed, a subsequent dose(s) after several months. Examples of suitable treatment schedules include: (i) 0, 1 month and 6 months, (ii) 0, 7 days and 1 month, (iii) 0 and 1 month, (iv) 0 and 6 months, or other schedules sufficient to elicit the desired responses expected to reduce disease symptoms, or reduce severity of disease.

The antibodies of the present invention may be used alone or in combination with other agents such as steroids (prednisone etc.), cyclophosphamide, cyclosporin A or a purine analogue (e.g. methotrexate, 6-mercaptopurine, or the like), or antibodies such as an anti-lymphocyte antigen antibody, an anti-leukocyte antigen antibody, a TNF antagonist e.g. an anti-TNF antibody or TNF inhibitor e.g. soluble TNF receptor, or agents such as NSAIDs or other cytokine inhibitors.

The present invention will now be described with reference to the following specific, non-limiting examples.

### Example 1

### Reagents and Assays

In order to select and characterize ATX-binding antibodies, constructs of human and mouse ATX were generated for mammalian cell expression. The three human ATX isoforms were produced using mammalian expression system with purification tag (GSHHHHHH, SEQ ID NO: 15) fused at the N-terminus of ATX at the propeptide cleavage site, which are: beta, the most abundant form in humans, SEQ ID NO: 11 residues 49-863; alpha, SEQ ID NO. 12 residues 49-915; and gamma, SEQ ID NO: 13 residues 49-888. Recombinant human ATX beta was used in most of the antibody characterization and was also biotinylated and used for phage panning. Both isoform beta and gamma were overexpressed easily and showed strong enzymatic activities.

In addition to human ATX beta isoform, recombinant mouse ATX beta isoform from R&D systems (UnitProt Q9R1E6, SEQ ID NO: 14, Cat No. 6187-EN-010) was also used in characterizing the antibodies.

### Assays Used

### ATX Inhibition Assay, Using the Fluorescent Autotaxin Substrate FS-3

The substrate FS-3 (Echelon Biosciences, Cat. No. L2000, U.S. Pat. No. 7,459,285) is a lysophospholipid-derivative which displays an increase in fluorescence when hydrolyzed by ATX, therefore activity can be measured by monitoring the rate of increase in fluorescence. Recombinant human autotaxin was diluted to 10 nM in assay buffer (50 mM Tris pH 8.0, 140 mM NaCl, 5 mM KCl, 1 mM CaCl₂, 1 mM MgCl₂, 0.1% fatty acid free BSA). The FS-3 substrate was diluted to 2 uM in assay buffer. Test mAbs were diluted to 10 μg/ml in assay buffer.

In a white 384 well plate, 15 ul autotaxin and 15 μl mAb were added in duplicate wells and incubated at room temperature for 10 minutes while shaking. Then 30 μl FS-3 substrate was added to each well. Final concentrations are 2.5 nM ATX, 2.5 μg/ml mAb, and 1 uM FS-3 in a volume of 60 μl.

Fluorescence (Excitation 485 nm, Emission 535 nm) was measured using a Molecular Devices Spectramax M2 plate reader in kinetic mode for 20 minutes with a 30 second interval, and the slope of the rate of change in fluorescence was calculated using Softmax Pro software. Controls included ATX in the absence of mAb, ATX with an isotype control mAb, and the FS-3 substrate in the absence of ATX. The rates are normalized to the rate of autotaxin hydrolysis of FS-3 with no inhibitor added to the reaction.

ATX Inhibition Assay of Autotaxin LPC Hydrolysis, by Detection of Choline with Amplex Red

ATX catalyzes the hydrolysis of LPC into LPA and choline. In this enzyme-coupled assay, ATX activity is monitored indirectly using 10-acetyl-3,7-dihydrophenoxazine (Amplex Red reagent), a sensitive fluorogenic probe for hydrogen peroxide. Choline is oxidized by choline oxidase to betaine and hydrogen peroxide, which reacts with Amplex Red reagent in the presence of horseradish peroxidase to generate a highly fluorescent product. ATX activity is measured by monitoring the rate of increase in fluorescence. The protocol was based on the manufacturer's instructions for the Amplex Red Phospholipase D Assay Kit (Invitrogen, Cat. No. A12219) with minor modifications. Autotaxin was diluted to 1.5 ug/ml in 1× Reaction Buffer. The supplied lecithin was substituted with lysophosphocholine (Sigma, L4129) at the same concentration. Each well contained 10 μL autotaxin (0.15 μg/ml or 1.5 nM) or buffer, 40 μl sample, and 50 μL Amplex Red working solution.

### Affinity Analysis

Affinity measurements using surface plasmon resonance (SPR) were performed using a Biacore 3000 optical biosensor (Biacore) for ATXB3, ATXB6, ATXB8, ATXB9, ATXB13, and ATXB14. A biosensor surface was prepared by coupling anti-Penta-His antibody (Qiagen, Cat No. 34660) to the carboxymethylated dextran surface of a CM-5 chip (Biacore, Cat No. BR-1000-14) using the manufacturer instructions for amine-coupling chemistry. Approximately 15,000 RU (response units) of anti-Penta-His antibody were immobilized in each of four flow cells. The kinetic experiments were performed at 25° C. in running buffer (PBS+0.01% P20+100 ug/ml BSA). Serial dilutions of anti-ATX mAbs (ATXB3, B6, B8, B9, B13, B14) from 900 nM to 0.412 nM were prepared in running buffer. About 80 RU of human ATX beta-His tagged proteins were captured on flow cell 2 of the sensor chip. Flow cell 1 was used as reference surface. Capture of ATX was followed by five minutes injection (association phase) of anti-ATX mAbs at 50 uL/min, followed by 15 minutes of buffer flow (dissociation phase). The chip surface was regenerated by 15 seconds injection of 100 mM H₃PO₄ (Sigma, Cat No. 7961) at 50 uL/min. The collected data were processed using BIAevaluation software, version 3.2 (Biacore). First, double reference subtraction of the data was performed by subtracting the curves generated by buffer injection from the reference-subtracted curves for analyte injections. Then kinetic analysis of the data was performed using the Bivalent Analyte binding model with global fitting. Kd1 (Kₒₙ) was calculated as intrinsic KD with minimum avidity. The result for each mAb was reported in the format of Ka1 (On-rate, Kₒₙ), Kd1 (Off-rate, K_{off}) and KD1 (Equilibrium dissociation constant). Affinity measurements for ATXB18 were performed on a Proteon XPR36 system (Bio-Rad), using a protocol similar to the one used on the Biacore platform.

### Bio-Layer Interferometry

Biomolecular interaction analysis using bio-layer interferometry were performed using an Octet RED384 instrument (Forte Bio). ATX was diluted to 20 μg/ml and mAbs to 10 μg/ml in PBSTB (PBS, 0.02% Tween, 0.1% BSA). Hexa-histidine-tagged ATX was bound to anti-Penta-His biosensors (ForteBio, Cat No. 18-0020) for 4 minutes, followed by a two minute primary mAb binding period then a two minute secondary mAb binding period. Biosensor traces were then analyzed qualitatively to determine if simultaneous binding occurred for each mAb pair.

### Example 2

### Selection of 57 ATX Binding Fab Clones

The de novo Fab-pIX libraries have been described Shi et al. J Mol Biol 397:385-396, 2010; WO09085462A1; U.S. Ser. No. 12/546,850; and herein above are designated 169, 323 and 551 which references the heavy-chain human germline framework being used: IGHV1-69 (SEQ ID NO: 1), IGHV3-23 (SEQ ID NO: 2), or IGHV5-51 (SEQ ID NO: 3) in IMGT nomenclature. The three heavy-chain library frameworks are combined with four light-chain library VLₖₐₚₚₐ frameworks: A27 (IGKV3-20*01 (SEQ ID NO: 5)), B3 (IGKV4-1*01 (SEQ ID NO: 6)), L6 (IGKV3-11*01 (SEQ ID NO: 7)), and O12 (IGKV1-39*01 (SEQ ID NO: 8)). In the libraries, the Fabs V-regions are completed by the addition of a J-region (FR4) comprising SEQ ID NO: 4 in the heavy chains and SEQ ID NO: 10 in the light chains. The heavy chain CDR3 is of variable length from 7-14 residues. Examples of the complete V-regions for each library are shown in FIG. 1 and numbered and CDR regions shown according to Kabat.

### Phage Panning

For selection of ATX binding Fabs, the de novo Fab libraries displayed on phage coat protein IX were panned against biotinylated human ATX beta following a published protocol for phage selections with soluble biotinylated antigens (Marks and Bradbury, Antibody Engineering. Humana Press, 248: 161-176, 2004). Briefly, in-house Fab-pIX de novo phage libraries and paramagnetic Streptavidin (SA) beads (Invitrogen, Cat. No. 112.05D) were blocked with 50% (v/v) ChemiBLOCKER (Millipore; Cat. No. 2170) in TBST (Tris buffered saline plus 0.01% Tween). The phage libraries were pre-adsorbed on blocked beads for 30 minutes to remove library components that react directly with the beads.

Fab-phage panning was carried out with biotinylated human ATX beta isoform at relatively high concentration of antigen and at low wash stringency during in the first round. The panning parameters were as follows: Round 1: 100 nM antigen, 1 hour incubation at room temperature, 5× washes with TBST followed by 1× wash with TBS; Round 2: 10 nM antigen, 1 hour incubation at room temperature, 10× washes with TBST/lx wash with TBS; Round 3: 1 nM antigen, 16 hour (overnight) incubation at 4° C., 10× washes with TBST/1× wash with TBS.

Biotinylated human ATX was added to the pre-absorbed libraries to a final concentration of 100 nM and incubated for 1 hour with gentle rotation. Blocked SA beads were added and incubated for 15 minutes to capture biotinylated ATX with bound phage. The magnetically-captured phage/antigen/bead complex was washed 5 times with 1 ml of TBST and once with 1 ml TBS. Following removal of the final TBS wash, 1 ml of exponentially growing TG1 cells (Stratagene, Cat. No. 200123) was added and incubated at 37° C. for 30 minutes without shaking. Infected bacteria were spread on LB/Agar (1% Glucose/100 μg/ml Carbenicillin) plates (Teknova, Cat. No. L5804) and incubated overnight at 37° C. Bacterial lawns were scraped and glycerol stocks prepared [15% Glycerol/Carbenicillin (100 μg/ml)/2×YT] and stored at −80° C. To prepare phage for second-round panning, 25 ml of 2×YT/Carbenicillin (100 μg/ml) was inoculated with 25 μl of bacterial glycerol stock and grown at 37° C. until an OD600 of roughly 0.5. Helper phage VCSM13 (Stratagene, Cat. No. 200251) was added to the culture at a multiplicity of infection of approximately 10:1 and incubation was carried out for 30 minutes at 37° C. without shaking. The bacteria was spun down and the pellet resuspended in induction media (2×YT/Carb/Kan/IPTG) and grown at 30° C. overnight. Phage was precipitated with 2% PEG/0.25M NaCl (final concentrations) and re-suspended in 2 ml of PBS. First-round phage was stored at 4° C. and used to carry out second-round panning. Antigen concentration was changed to 10 nM and 1 nM for the second-round and third-round panning, respectively.

### Monoclonal Soluble Fab-His Binding ELISAs

Primary ELISA screening of individual clones was performed using secreted soluble Fab-His protein from E. coli supernatants. In the pIX phage display vector (pCNTO-Fab-pIX), the Fab CH 1 domain is fused in-frame to the pIX phage-coat protein. Restriction enzyme digestion of the vector with NheI and SpeI results in excision of the pIX gene. Self-ligation/re-circularization of the resulting linearized pIX-minus plasmid leads to the in-frame fusion of a 6×His-tag to the CH1 Fab domain. Purified pIX-minus plasmid was self-ligated/re-circularized and used to transform TG1 cells. Bacterial supernatants, from fresh cultures containing soluble Fab-His protein, were used to carry out binding ELISAs.

Fab-His protein prepared as described above was used in a primary binding ELISA screen with biotinylated human ATX beta at a concentration of 5 nM, which should allow the detection of clones with affinities in the nanomolar affinity range. Fabs were captured on black MaxiSorp plates (Nunc, Cat. No. 437111) coated with 1 μg/ml sheep anti-human Fd (CH1) antibody (The Binding Site, Cat. No. PC075). Plates were washed and biotinylated human ATX was added to the captured Fabs at 5 nM. Incubation was carried out for 1 hour at room temperature with gentle shaking. Plates were washed, SA-HRP (Invitrogen, Cat. No. 43-4323) was added and chemiluminescent detection carried out.

Clones with binding above background were sequenced and unique clones identified. Table 3 shows the 57 unique Fabs which compositions and the identified germline origin for each pair of variable regions (VH and VL). These clones were identified with binding signal to human ATX at least 6-fold above the background. Table 3 lists the identifier for VH and VL (Peptide ID) and the combination of both (Fab ID) sorted by the Fab ELISA binding data.

TABLE 3
57 ATX-binding Fab clones

| | Fab | | VH- | | VL- |
|---|---|---|---|---|---|
| Fab ID | Binding | VH | Origin | VL | Origin |
| F17 | 2.4E+07 | H11 | 551 | L14 | L6 |
| F73 | 1.7E+07 | H66 | 169 | L67 | O12 |
| F67 | 8.8E+06 | H60 | 169 | L61 | O12 |
| F40 | 8.1E+06 | H32 | 323 | L42 | L6 |
| F96 | 5.9E+06 | H89 | 169 | IFWL124 | O12 |
| F97 | 5.8E+06 | H90 | 169 | IFWL124 | O12 |
| F89 | 5.7E+06 | H70 | 169 | L82 | A27 |
| F104 | 5.1E+06 | H96 | 169 | IFWL124 | O12 |
| F143 | 4.9E+06 | H148 | 169 | H2RL12 | O12 |
| F86 | 4.2E+06 | H80 | 169 | L80 | O12 |
| F16 | 3.9E+06 | H10 | 551 | L13 | O12 |
| F5 | 3.8E+06 | H4 | 551 | L4 | L6 |
| F118 | 2.6E+06 | H111 | 169 | L104 | A27 |
| F126 | 2.4E+06 | H123 | 169 | L110 | A27 |
| F53 | 2.4E+06 | H2 | 169 | L30 | L6 |
| F34 | 2.3E+06 | H2 | 169 | L28 | L6 |
| F130 | 2.2E+06 | H129 | 169 | L113 | A27 |
| F35 | 2.1E+06 | H25 | 169 | L29 | L6 |
| F69 | 2.0E+06 | H63 | 169 | L63 | L6 |
| F32 | 2.0E+06 | H7 | 169 | PH9L4 | O12 |
| F135 | 2.0E+06 | H138 | 169 | L118 | A27 |
| F50 | 1.9E+06 | H25 | 169 | L46 | L6 |
| F122 | 1.9E+06 | H119 | 169 | L108 | A27 |
| F100 | 1.9E+06 | H93 | 169 | PH9L1 | A27 |
| F116 | 1.9E+06 | H108 | 169 | L100 | O12 |
| F78 | 1.9E+06 | H73 | 169 | L73 | A27 |
| F128 | 1.8E+06 | H127 | 169 | L111 | A27 |
| F68 | 1.8E+06 | H62 | 169 | L62 | A27 |
| F117 | 1.8E+06 | H110 | 169 | L103 | O12 |
| F7 | 1.8E+06 | H2 | 169 | L6 | L6 |
| F11 | 1.8E+06 | H2 | 169 | L9 | L6 |
| F90 | 1.7E+06 | H83 | 169 | L83 | A27 |
| F71 | 1.7E+06 | H2 | 169 | L65 | L6 |
| F74 | 1.7E+06 | H68 | 169 | L68 | L6 |
| F85 | 1.7E+06 | H58 | 169 | L79 | A27 |
| F8 | 1.6E+06 | H2 | 169 | L7 | L6 |
| F12 | 1.6E+06 | H7 | 169 | L10 | L6 |
| F62 | 1.6E+06 | H54 | 169 | GCGL18 | O12 |
| F93 | 1.6E+06 | H86 | 169 | L79 | A27 |
| F72 | 1.6E+06 | H65 | 169 | PH9L1 | A27 |
| F2 | 1.5E+06 | H2 | 169 | L1 | L6 |
| F123 | 1.4E+06 | H120 | 169 | PH9L4 | O12 |
| F121 | 1.4E+06 | H115 | 169 | L106 | L6 |
| F3 | 1.4E+06 | H2 | 169 | L2 | L6 |
| F142 | 1.3E+06 | H146 | 169 | L121 | O12 |
| F102 | 1.3E+06 | H2 | 169 | L91 | L6 |
| F133 | 1.2E+06 | H134 | 169 | L117 | A27 |
| F144 | 1.2E+06 | H149 | 169 | L122 | A27 |
| F45 | 1.2E+06 | H2 | 169 | L34 | L6 |
| F33 | 1.1E+06 | H24 | 323 | L35 | O12 |
| F13 | 1.1E+06 | H2 | 169 | L11 | L6 |
| F114 | 1.1E+06 | H106 | 169 | L99 | L6 |
| F9 | 1.0E+06 | H2 | 169 | L8 | L6 |
| F54 | 9.8E+05 | H44 | 323 | IFWL195 | O12 |
| F63 | 9.8E+05 | H55 | 169 | L57 | L6 |
| F43 | 9.8E+05 | H2 | 169 | L44 | L6 |
| F105 | 9.7E+05 | H97 | 169 | L93 | A27 |

Sequencing of Fab Clone Hits from Primary ELISA Screen

Individual overnight bacterial cultures corresponding to human ATX binding Fabs identified by the antigen-binding ELISA screen described above were used as a template for PCR amplification of Fab cassettes. Amplification of the Fab DNA (VH, CH, VL, CL) results in a 1.7 kb DNA fragment which is sequenced with multiple forward and reverse primers using standard protocols. Sequence files were analyzed with the SeqScape software (Applied Biosystems).

### Results

Based on the germline origins and their binding activity to human ATX as Fabs, the 12 highest affinity binding Fab from VH germline 1-69, and the top three binding Fabs from VH germline 3-23 and 5-51 were selected for further characterization. All 18 Fabs were converted to mAbs with human IgG1/human Kappa constant regions. Table 4 shows the paring of each recombinant mAb ID and their VH and VL pairing. The 18 VH-VL pairs represented 17 unique VH and 16 unique VL.

TABLE 4

| mAb | FAB | VH SEQ | VH.Peptide | VL SEQ | VL.Peptide | |
|---|---|---|---|---|---|---|
| AA ID | ID | ID | ID | ID | ID | Activity |
| B1 | F5 | 16 | H4 | 17 | L4 | Binder |
| B2 | F16 | 18 | H10 | 19 | L13 | Binder |
| B3 | F17 | 20 | H11 | 21 | L14 | Binder |
| B4 | F53 | 22 | H2 | 23 | L30 | Binder |
| B5 | F34 | 22 | H2 | 25 | L28 | Binder |
| B6 | F73 | 24 | H66 | 27 | L67 | Binder & Neutralizer |
| B7 | F86 | 26 | H80 | 29 | L80 | Binder & Neutralizer |
| B8 | F96 | 28 | H89 | 31 | IFWL124 | Binder & Neutralizer |
| B9 | F89 | 30 | H70 | 33 | L82 | Binder & Neutralizer |
| B10 | F67 | 32 | H60 | 35 | L61 | Binder |
| B11 | F118 | 34 | H111 | 37 | L104 | Binder |
| B12 | F126 | 36 | H123 | 39 | L110 | Binder |
| B13 | F143 | 38 | H148 | 41 | H2RL12 | Binder & Neutralizer |
| B14 | F104 | 40 | H96 | 31 | IFWL124 | Binder & Neutralizer |
| B15 | F33 | 42 | H24 | 43 | L35 | Binder |
| B16 | F40 | 44 | H32 | 45 | L42 | Binder |
| B17 | F54 | 46 | H44 | 47 | IFWL195 | Binder |
| B18 | F97 | 48 | H90 | 31 | IFWL124 | Binder & Neutralizer |

### Example 3

### Characterization of ATX Binding Mabs

One of the objectives of the research was to select high affinity binders to ATX capable of blocking the enzymatic activity of ATX.

Of the 57 initially selected ATX-binding Fabs, 18 were cloned into vectors for conversion to full-length human IgG1 Mabs. Routine procedures were used to express and purify the disclosed antibodies. DNA encoding the mAbs molecules were constructed from the Fab clones by fusing VH from Fab with human IgG1 isotype and VL with human Kappa using standard molecule cloning techniques. The heavy and light chains were transiently expressed in HEK 293F cells. The harvested supernatants were purified via Protein A chromatography.

Once sufficient material was available, the characterization assays were preformed, including:

(1) ability to block lysophoslipase activity as measured using the FS-3 assay,
(2) IC50 in the lysophosphocholine cleavage assay,
(3) affinity by SPR, and
(4) competitive binding analysis or “binning”.

### Results

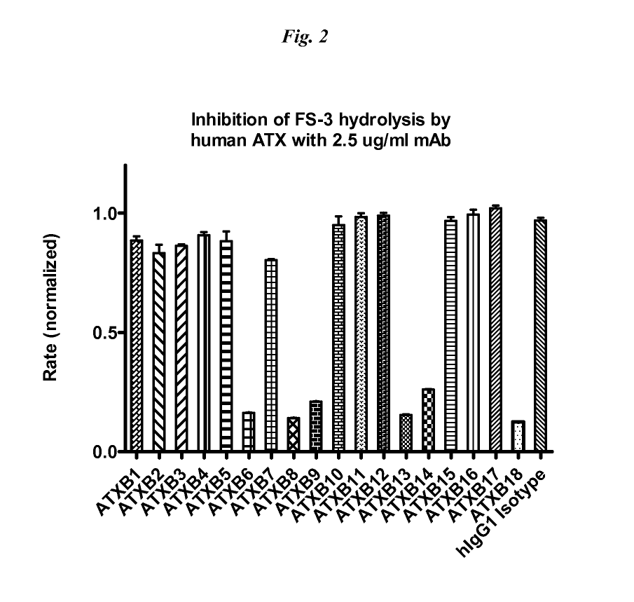

The eighteen converted mAbs were first tested in a single-point ATX inhibition assay using the fluorescent substrate, FS-3. The results of this assay, where the measured reaction rate in the presence of each antibody has been normalized to the reaction rate of ATX in the absence of antibody, are shown in FIG. 2. Six variants (B6, B8, B9, B13, B14, and B18) showed significant inhibition of substrate hydrolysis by ATX beta isoform and were selected for further characterization. A seventh (B7) which showed lesser inhibition at this concentration, so was also selected. A human IgG1 isotype control antibody showed no inhibition of in this assay. Similar inhibition were observed when B7, B8, and B9 were tested in the same assay using 0.5 μM of either human ATX beta or gamma isoforms, indicating the activity was not isoform specific.

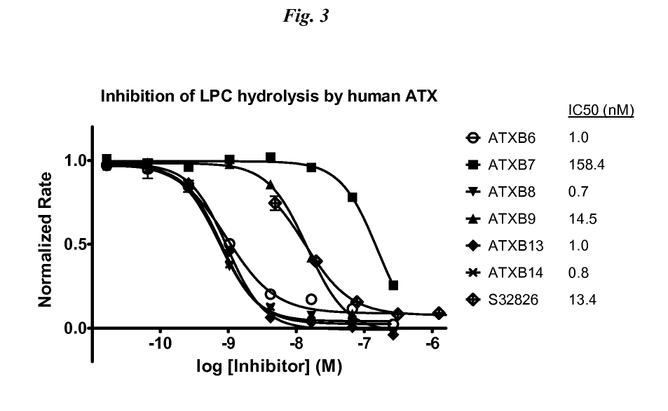

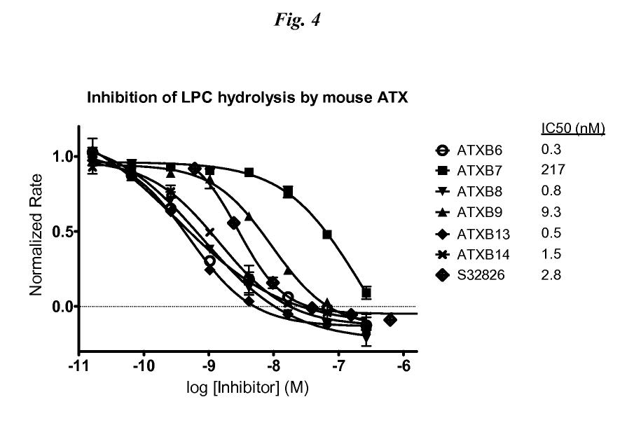

Six mAbs (B6, B7, B8, B9, B13, and B14) were selected and tested at varying concentrations in another ATX inhibition assay that measures hydrolysis of the natural ATX substrate, LPC. The IC50 values, were calculated from a four-parameter dose-response model from the data shown in FIGS. 3 and 4 for each variant tested for inhibition of human and mouse ATX respectively. All variants displayed a dose-dependent inhibition of ATX conversion of LPC to LPA and choline. The small molecule autotaxin inhibitor, 532826, was included as a reference point. The hIgG1 isotype control antibody CNTO 3930 did not inhibit autotaxin in these assays (data not shown). The mAb ATXB18 had not yet been produced when these assays were conducted and was not yet tested in this assay.

The five mAbs with the lowest IC50 values in the inhibition assays as well as ATXB3, a non-neutralizing antibody, were tested for their affinity to the human ATX-beta isoform, as measured by surface plasmon resonance. Due to nonspecific binding of the ATX antigen to the chip surface, these experiments had to be conducted in the reverse format, with ATX immobilized on the chip surface (ligand) and our antibodies in solution (analyte). Table 5 lists the measured association rate (ka1, Kₒₙ), dissociation rate (kd1, k_{off}), and dissociation constant (KD1) for each mAb to ATX, calculated using the bivalent analyte model where E stands for base 10.

TABLE 5

| mAb IDs | ka1 (1/Ms) | kd1 (1/s) | KD1 (M) |
|---|---|---|---|
| ATXB3 | 2.12E+05 | 3.03E−04 | 7.15E−10 |
| ATXB6 | 4.44E+05 | 2.92E−04 | 3.29E−10 |
| ATXB8 | 2.49E+05 | 8.83E−04 | 1.77E−09 |
| ATXB9 | 3.37E+03 | 3.22E−04 | 4.78E−08 |
| ATXB13 | 2.74E+05 | 3.29E−04 | 6.00E−10 |
| ATXB14 | 7.89E+05 | 5.83E−04 | 3.69E−10 |
| ATXB18 | 9.49E+05 | 6.14E−04 | 6.73E−10 |

Finally, real-time monitoring of the antibody-ATX biomolecular interaction using Bio-Layer Interferometry on an Octet Red384 instrument was used to determine which of 18 antibodies could bind to ATX simultaneously, and which could not due to epitope overlap, steric hindrance or other reason. A subset of the possible mAb pairings were tested with two goals in mind: (1) to determine whether or not the neutralizing mAbs bound to the same epitope and (2) to identify mAbs that are able to bind ATX simultaneously and, especially, at the same time that a neutralizing mAb was bound.

In this assay, the primary mAb was bound to ATX first, and the complex probed for the ability of the secondary mAb to bind. Table 6 shows the compiled results of the mAb pairs tested with a “Y” or “N” indicating whether or not simultaneous binding is possible. An empty box indicates that this pair has not been tested, while a black box denotes self-competition. The neutralizing mAbs exhibiting the highest binding affinity (ATXB6, ATXB8, ATXB13, and ATXB14) were not able to bind ATX simultaneously suggesting that their epitopes may overlap. However, the weaker affinity mAb, ATXB7, was able to bind simultaneously with some of the other neutralizing mAbs and thus binds to a different epitope. In addition, a number of non-neutralizing mAbs were found to occupy different epitopes from those of the neutralizing mAbs, including ATXB2, ATXB3, ATXB10, ATXB15, and ATXB16. ATXB10, ATXB15, and ATXB16 appear to compete for the same epitope.

TABLE 6
Screening mAb pairs for simultaneous binding to ATX.

| | | Secondary mAb | | | | | | | | | | | | | | | | | |
|---|---|---|---|---|---|---|---|---|---|---|---|---|---|---|---|---|---|---|---|
| Simultaneous | | ATX | ATX | ATX | ATX | ATX | ATX | ATX | ATX | ATX | ATX | ATX | ATX | ATX | ATX | ATX | ATX | ATX | ATX |
| Binding? | | B1 | B2 | B3 | B4 | B5 | B6 | B7 | B8 | B9 | B10 | B11 | B12 | B13 | B14 | B15 | B16 | B17 | B18 |
| Primary | ATXB1 | 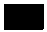 | | | | | | | | | | | | | | | | | |
| mAb | | | | | | | | | | | | | | | | | | | |
| | ATXB2 | | 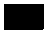 | Y | | | | Y | | | Y | | | | | Y | Y | | |
| | ATXB3 | | Y | 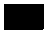 | | | Y | Y | Y | | Y | | | Y | | Y | Y | | |
| | ATXB4 | | | | 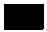 | | | | | | | | | | | | | | |
| | ATXB5 | | | | | 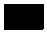 | | | | | | | | | | | | | |
| | ATXB6 | N | Y | Y | N | N | 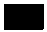 | Y | N | N | Y | N | N | N | N | Y | Y | N | N |
| | ATXB7 | | Y | Y | | | Y | 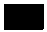 | Y | | Y | | | Y | | Y | Y | | |
| | ATXB8 | N | Y | Y | N | N | N | Y | 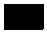 | N | Y | N | N | N | N | Y | Y | N | N |
| | ATXB9 | | | | | | | | | 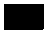 | | | | | | | | | |
| | ATXB10 | | Y | Y | | | | Y | | | 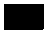 | | | | | N | N | | |
| | ATXB11 | | | | | | | | | | | 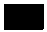 | | | | | | | |
| | ATXB12 | | | | | | | | | | | | 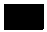 | | | | | | |
| | ATXB13 | N | Y | Y | N | N | N | Y | N | N | Y | N | N | 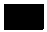 | N | Y | Y | N | N |
| | ATXB14 | N | Y | Y | N | N | N | Y | N | N | Y | N | N | N | 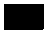 | Y | Y | N | N |
| | ATXB15 | | Y | Y | | | | Y | | | N | | | | | 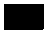 | N | | |
| | ATXB16 | | Y | Y | | | | Y | | | N | | | | | N | 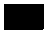 | | |
| | ATXB17 | | | | | | | | | | | | | | | | | 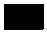 | |
| | ATXB18 | | | | | | | | | | | | | | | | | | 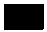 |

### Summary

Among the 18 mAbs tested, seven were categorized as neutralizers by demonstrating the ability to inhibit ATX hydrolytic activities. The neutralizers have binding affinity to human ATX in the range of 300 pM to 48 nM. Groups of antibodies with overlapping and non-overlapping epitopes on ATX were also identified (Table 7).

TABLE 7

| | | When pre-bound to | Cannot bind ATX |
|---|---|---|---|
| | | ATX, Mab prevents | when one of |
| mAb | | binding of specified | specified Mabs |
| AA ID | Activity | Mabs to ATX | pre-bound |
| B1 | Binder | No data | B6, B8, B13, B14 |
| B2 | Binder | None as tested | No data |
| B3 | Binder | None as tested | No data |
| B4 | Binder | No data | B6, B8, B13, B14 |
| B5 | Binder | No data | B6, B8, B13, B14 |
| B6 | Binder & | B1, B4, B5, B8, B9, | B8, B13, B14 |
| | Neutralizer | B11-14, B17, B18 | |
| B7 | Binder & | None | No data |
| | Neutralizer | | |
| B8 | Binder & | B1, B4-6, B9, B11-14, | B6, B8, B13, B14 |
| | Neutralizer | B17, B18 | |
| B9 | Binder & | No data | B6, B8, B13, B14 |
| | Neutralizer | | |
| B10 | Binder | B15, B16 | B15, B16 |
| B11 | Binder | No data | B6, B8, B13, B14 |
| B12 | Binder | No data | B6, B8, B13, B14 |
| B13 | Binder & | B1, B4-6, B8, B9, B11, | B6, B8, B14 |
| | Neutralizer | B12, B14, B17, B18 | |
| B14 | Binder & | B1, B4-6, B8, B9, | B6, B8, B13 |
| | Neutralizer | B11-13, B17, B18 | |
| B15 | Binder | B10 | B10, B16 |
| B16 | Binder | B10 | B10, B15 |
| B17 | Binder | No data | B6, B8, B13, B14 |
| B18 | Binder & | No data | B6, B8, B13, B14 |
| | Neutralizer | | |

### Example 4

### Structure Activity Relationship

Table 8A-B show the V-region CDR identified for the 57 Fabs clones with a unique pair of variable heavy and light chains (VH and VL). These clones were identified with binding signal to human ATX at least 6-fold above the background.

The CDRs of each VH and VL sequence following Kabat CDR definition (Kabat et al., 5th edit. Public Health Service, NIH, Washington, D.C., 1991; Wu and Kabat, J. Exp. Med 132:211-250, 1970) were identified. Each unique CDR sequence is listed by SEQ ID Table 8A (VH CDRs) and 8B (VL CDRs). Note that the VH designated H2 of germline 1-69 origin with H-CDR1=SEQ ID 49, H-CDR2=SEQ ID NO: 53 and H-CDR3=SEQ ID NO: 69 was present 11 times.

TABLE 8A
Heavy Chain Variable CDR sequences for the 57 Fab clones.

| Fab ID | | | CDR-H1 | CDR-H2 | CDR-H3 |
|---|---|---|---|---|---|
| (Mab ID) | VH | VH-GL | SEQ ID | SEQ ID | SEQ ID |
| F73 (B6) | H66 | 1-69 | 49 | 53 | 61 |
| F96 (B8) | H89 | 1-69 | 49 | 53 | 62 |
| F97 (B18) | H90 | 1-69 | 49 | 53 | 63 |
| F104 (B14) | H96 | 1-69 | 49 | 54 | 64 |
| F143 (B13) | H148 | 1-69 | 49 | 53 | 65 |
| F86 (B7) | H80 | 1-69 | 49 | 53 | 66 |
| F89 (B9) | H70 | 1-69 | 49 | 55 | 67 |
| F67 (B10) | H60 | 1-69 | 49 | 53 | 68 |
| P53 (B4) | H2 | 1-69 | 49 | 53 | 69 |
| F34 (B5) | H2 | 1-69 | 49 | 53 | 69 |
| F118 (B11) | H111 | 1-69 | 50 | 53 | 70 |
| F126 (B12) | H123 | 1-69 | 49 | 53 | 71 |
| F33 (B15) | H24 | 3-23 | 51 | 56 | 72 |
| F54 (B17) | H44 | 3-23 | 51 | 56 | 73 |
| F40 (B16) | H32 | 3-23 | 51 | 57 | 74 |
| P16 (B2) | H10 | 5-51 | 52 | 58 | 75 |
| F17 (B3) | H11 | 5-51 | 52 | 58 | 76 |
| F5 (B1) | H4 | 5-51 | 52 | 58 | 77 |
| F32 | H7 | 1-69 | 49 | 53 | 78 |
| F116 | H108 | 1-69 | 49 | 53 | 79 |
| F117 | H110 | 1-69 | 49 | 53 | 80 |
| F62 | H54 | 1-69 | 49 | 53 | 81 |
| F123 | H120 | 1-69 | 49 | 53 | 82 |
| F142 | H146 | 1-69 | 49 | 53 | 83 |
| F35 | H25 | 1-69 | 49 | 53 | 84 |
| F69 | H63 | 1-69 | 49 | 53 | 85 |
| F50 | H25 | 1-69 | 49 | 53 | 84 |
| F7 | H2 | 1-69 | 49 | 53 | 69 |
| F11 | H2 | 1-69 | 49 | 53 | 69 |
| F71 | H2 | 1-69 | 49 | 53 | 69 |
| F74 | H68 | 1-69 | 49 | 53 | 86 |
| F8 | H2 | 1-69 | 49 | 53 | 69 |
| F12 | H7 | 1-69 | 49 | 53 | 78 |
| F2 | H2 | 1-69 | 49 | 53 | 69 |
| F121 | H115 | 1-69 | 49 | 53 | 87 |
| F3 | H2 | 1-69 | 49 | 53 | 69 |
| F102 | H2 | 1-69 | 49 | 53 | 69 |
| F45 | H2 | 1-69 | 49 | 53 | 69 |
| F13 | H2 | 1-69 | 49 | 53 | 69 |
| F114 | H106 | 1-69 | 49 | 59 | 88 |
| F9 | H2 | 1-69 | 49 | 53 | 69 |
| F63 | H55 | 1-69 | 50 | 53 | 89 |
| F43 | H2 | 1-69 | 49 | 53 | 69 |
| F130 | H129 | 1-69 | 49 | 53 | 90 |
| F135 | H138 | 1-69 | 49 | 53 | 91 |
| F122 | H119 | 1-69 | 49 | 53 | 92 |
| F100 | H93 | 1-69 | 49 | 54 | 93 |
| F78 | H73 | 1-69 | 49 | 53 | 94 |
| F128 | H127 | 1-69 | 49 | 59 | 95 |
| F68 | H62 | 1-69 | 50 | 60 | 96 |
| F90 | H83 | 1-69 | 49 | 53 | 97 |
| F85 | H58 | 1-69 | 49 | 53 | 98 |
| F93 | H86 | 1-69 | 49 | 53 | 99 |
| F72 | H65 | 1-69 | 49 | 53 | 100 |
| F133 | H134 | 1-69 | 49 | 53 | 101 |
| F144 | H149 | 1-69 | 50 | 53 | 102 |
| F105 | H97 | 1-69 | 50 | 53 | 103 |

Table 8B gives the VL compositions of the 57 Fabs designated by germline origin and individual L-CDR. The VL designated IFWL124 (SEQ ID NO: 31) of O12 origin was found in three Fabs, all of which F96 (Mab B8), F97 (B18), and F104 (B14) were found to neutralize ATX catalytic activity. Three Fabs had also had the germline A27 VL and L-CDRs (PH9L1) and two had the closely related A27 VL designated (L79), however, none of these Fabs were tested for neutralization.

TABLE 8B
VL CDR sequences for the 57 Fab clones.

| | | | L-CDR1 | L-CDR2 | L-CDR3 |
|---|---|---|---|---|---|
| Fab ID | VL | VL-GL | SEQ ID | SEQ ID | SEQ ID |
| F73 (B6) | L67 | O-12 | 104 | 139 | 155 |
| F96 (B8) | IFWL124 | O-12 | 105 | 140 | 156 |
| F97 (B18) | IFWL124 | O-12 | 105 | 140 | 156 |
| F104 (B14) | IFWL124 | O-12 | 105 | 140 | 156 |
| F143 (B13) | H2RL12 | O-12 | 106 | 140 | 156 |
| F86 (B7) | L80 | O-12 | 106 | 141 | 156 |
| F89 (B9) | L82 | A-27 | 107 | 142 | 157 |
| F67 (B10) | L61 | O-12 | 108 | 140 | 158 |
| F53 (B4) | L30 | L-6 | 109 | 143 | 159 |
| F34 (B5) | L28 | L-6 | 110 | 143 | 160 |
| F118 (B11) | L104 | A-27 | 111 | 144 | 161 |
| F126 (B12) | L110 | A-27 | 111 | 144 | 162 |
| F33 (B15) | L35 | O-12 | 112 | 140 | 156 |
| F54 (B17) | IFWL195 | O-12 | 113 | 140 | 156 |
| F40 (B16) | L42 | L-6 | 114 | 145 | 163 |
| F16 (B2) | L13 | O-12 | 115 | 140 | 156 |
| F17 (B3) | L14 | L-6 | 114 | 143 | 164 |
| F5 (B1) | L4 | L-6 | 116 | 146 | 165 |
| F32 | PH9L4 | O-12 | 108 | 140 | 156 |
| F116 | L100 | O-12 | 117 | 147 | 166 |
| F117 | L103 | O-12 | 118 | 140 | 167 |
| F62 | GCGL18 | O-12 | 108 | 148 | 156 |
| F123 | PH9L4 | O-12 | 108 | 140 | 156 |
| F142 | L121 | O-12 | 108 | 140 | 168 |
| F35 | L29 | L-6 | 110 | 143 | 169 |
| F69 | L63 | L-6 | 119 | 146 | 170 |
| F50 | L46 | L-6 | 120 | 149 | 171 |
| F7 | L6 | L-6 | 121 | 146 | 172 |
| F11 | L9 | L-6 | 122 | 149 | 173 |
| F71 | L65 | L-6 | 123 | 150 | 174 |
| F74 | L68 | L-6 | 124 | 151 | 175 |
| F8 | L7 | L-6 | 125 | 152 | 176 |
| F12 | L10 | L-6 | 126 | 152 | 177 |
| F2 | L1 | L-6 | 127 | 150 | 178 |
| F121 | L106 | L-6 | 128 | 149 | 179 |
| F3 | L2 | L-6 | 129 | 152 | 180 |
| F102 | L91 | L-6 | 130 | 143 | 181 |
| F45 | L34 | L-6 | 131 | 146 | 182 |
| F13 | L11 | L-6 | 130 | 145 | 183 |
| F114 | L99 | L-6 | 132 | 146 | 184 |
| F9 | L8 | L-6 | 133 | 149 | 185 |
| F63 | L57 | L-6 | 134 | 143 | 186 |
| F43 | L44 | L-6 | 110 | 143 | 187 |
| F130 | L113 | A-27 | 111 | 144 | 188 |
| F135 | L118 | A-27 | 111 | 144 | 189 |
| F122 | L108 | A-27 | 111 | 144 | 190 |
| F100 | PH9L1 | A-27 | 111 | 144 | 191 |
| F78 | L73 | A-27 | 111 | 144 | 192 |
| F128 | L111 | A-27 | 111 | 144 | 193 |
| F68 | L62 | A-27 | 111 | 144 | 194 |
| F90 | L83 | A-27 | 135 | 153 | 195 |
| F85 | L79 | A-27 | 111 | 144 | 196 |
| F93 | L79 | A-27 | 111 | 144 | 196 |
| F72 | PH9L1 | A-27 | 111 | 144 | 191 |
| F133 | L117 | A-27 | 136 | 154 | 197 |
| F144 | L122 | A-27 | 137 | 153 | 198 |
| F105 | L93 | A-27 | 138 | 142 | 199 |

Based on the mAb characterization described in Example 3, the sequences of the 18 mAbs listed in Table 4 are compared with the sequences of the V-regions from all of the 57 unique binding pairs.

Among the binding Fab clones:
of those clones with the highest binding affinity (in relative chemiluminescence units, See Table 3) the heavy chain germline origins were predominantly 1-69 and the light chain origin was IGVK1-39 (O12).
A specific V-region (H2) was found in 13 Fabs each having a unique L6-derived VL.
All seven neutralizing mAbs B6, 7, 8, 9, 13, 14 and 18 (corresponding to Fabs F73, F86, F96, F89, F143, F104, and F97, respectively) are of VH 1-69 and, B6, 7, 8, 13, 14 and 18 have VL of O12 origin. All but B9 have the motif YGX₅X₆DY where X₅ is D or F, and X₆ is F or L in H-CDR3 (residues 103-108 of SEQ ID NO: 200).
   Mab B9 (Fab F89) bound ATX with high affinity and is a neutralizer, is from VH germline 1-69 but does not have the identified motif (YGX₅X₆DY where X₅ is D or F, and X₆ is F or L in H-CDR3 (residues 103-108 of SEQ ID NO: 200)) in H-CDR3 and the VL is from A27.
   Mab B10 (Fab F67) was one of the top three binders to ATX, has VH germline 1-69 frameworks and VL germline IGVK1-39 (O-12) frameworks, however, it has a longer H-CDR3 which does not contain the formula (YGX₅X₆DY where X₅ is D or F, and X₆ is F or L in H-CDR3 (residues 103-108 of SEQ ID NO: 200)) and it is also not a neutralizer.
   Mab B3 (F17) contains H-CDR3 PYGTFDY (SEQ ID NO: 76), is of VH5-51 and VL L6 origin and is not a neutralizer.
For the six neutralizing Mabs having a VL of O12 origin, three have the identical VL (IFW124, SEQ ID NO: 31) and five have the identical L-CDR3 (QQSYSTPL, SEQ ID NO: 156).

Therefore, a consensus sequence for an ATX neutralizing human IGHV1-69 germline derived VH comprising FR1-CDR1-FR2-CDR2-FR3-CDR3 can be represented as SEQ ID NO: 200:

QVQLVQSGAEVKKPGSSVKVSCKASGGTFSSYAISWVRQAPGQGLEWMG
GI X₁P X₂FG X₃ X₄NYAQKFQGRVTITADESTSTAYMELSSLRSED
TAVYYCARDLYIYG X₅ X₆DY

wherein X₁ is I or S, X₂ is I or Y, X₃ is N or T, X₄ is A or T, X₅ is D or

F and X₅ is F or L.

As six of seven VL derive from the IGVK1-39 (012) germline (L67, L80, IFWL124, or L82, SEQ ID NO: 27, 29, 31, or 33), a consensus sequence for an ATX neutralizing human gene derived VL comprising FR1-CDR1-FR2-CDR2-FR3-CDR3 can be represented as SEQ ID NO: 201:

DIQMTQSPSSLSASVGDRVTITCRASQSI X₁ X₂WLNWYQQKPGKAPKL
LIYX₃ASSLQSGVPSRFSGSGSGTDFTLTISSLQPEDFATYYCQQSYX₄
X₅P X₆T

wherein X₁ is A, D or G; X₂ is G or K; and X₃ is A or T; and X₄ is S or T; X₅ is T or Y; and X₆ is I or L.

Based on these analyses, an antibody or antibody fragment comprising binding domains derived from the human germline VH 1-69 combined with human germline VLkappa O12 has a high likelihood of specific, high affinity binding to ATX. In particular embodiment, an antibody or antibody fragment comprising one of the four variants of SEQ ID NO: 200 and a human germline VL kappa O12-derived would be expected to bind ATX and be capable of neutralizing ATX catalytic activity.

Sequence Table

| SEQ ID | | |
|---|---|---|
| NO: | Description | SEQUENCE AND FEATURES |
| 1 | Human IGHV1- | QVQLVQSGAE VKKPGSSVKV SCKASGGTFS SYX₁ISWVRQA |
| | 69*01 | PGQGLEWMGX₂ IX₃X₄X₅X₆GTANY AQKFQGRVTI TADESTSTAY |
| | | MELSSLRSED TAVYYCAR |
| | | Where X₁ is A or G, X₂ is G or W, X₃ may be I |
| | | or S, X₄ may be P or A, X₅ may be I or Y and X₆ |
| | | may be F or N. |
| 2 | Human IGHV3- | EVQLLESGGG LVQPGGSLRL SCAASGFTFS X₁YX₂MX₃WVRQA |
| | 23*01 | PGKGLEWVSX₄ IX₅X₆X₇GX₈STYY ADSVKGRFTI SRDNSKNTLY |
| | | LQMNSLRAED TAVYYCAK |
| | | Where X₁ may be S, D, N, or T; X₂ may be A, G, |
| | | or W; X₃ may be S or H; X₄ may by V, A, N or G; |
| | | X₅ may be S, N, K or W; X₆ may be Y, S, G, or Q; |
| | | X₇ may be S or D; and X₈ may be S or G |
| 3 | Human IGHV5- | EVQLVQSGAE VKKPGESLKI SCKGSGYSFT X₁YWIX₂WVRQM |
| | 51*01 | PGKGLEWMGX₃ IX₄PX₅DSX₆TRY SPSFQGQVTI SADKSISTAY |
| | | LQWSSLKASD TAMYYCAR |
| | | Where X₁ may be S, N, or T; X₂ may be S or G; X₃ |
| | | may be I or R; X₄ may by D or Y; X₅ may be G or |
| | | S; X₆ may be D or Y |
| 4 | Human JH | WGQGTLVTVSS |
| 5 | Human IGKV1- | DIQMTQSPSS LSASVGDRVT ITCRASQSI X₁ X₂X₃LNWYQQKP |
| | 39*01 (O12) | GKAPKLLIYX₄ ASSLQSGVPS RFSGSGSGTD FTLTISSLQP |
| | | EDFATYYC |
| | | Where X₁ may be A, D, N, S, or R; X₂ may be D, |
| | | G, K, N, or S; X₃ may be A, D, F, H, N, S, W, |
| | | V, or Y; X₄ may be A, D, F, G, K, N, T, or Y. |
| 6 | IGKV3-11 (L6) | EIVLTQSPAT LSLSPGERAT LSCRASQSV X₁X₂X₃LAWYQQKP |
| | | GQAPRLLIYX₄ ASNRATGIPA RFSGSGSGTD FTLTISSLEP |
| | | EDFAVYYC |
| | | Where X₁ may be A, D, N, R or S; X₂ may be D, |
| | | K, N, or S; X₃ may be A, D, F, H, N, S, W, or |
| | | Y; X₄ may be A, D, K, G, F, T or N. |
| 7 | Human IGKV3- | EIVLTQSPGT LSLSPGERAT LSCX₁X₂X₃X₄SVS SSYLAWYQQK |
| | 20 (A27) | PGQAPRLLIY X₅ASSRATGIP DRFSGSGSGT DFTLTISRLE |
| | | PEDFAVYYC |
| | | Where X₁ may be D, N, S, R or T; X₂ may be N, S, |
| | | or R; X₃ may be A, D, H, N, S, or R; X₄ may be |
| | | E, F, H, K, Q, S, or Y; X₅ may be A, D, G, or |
| | | S. |
| 8 | Human IGKV4- | DIVMTQSPDS LAVSLGERAT INCKSSQSVL X₁SSNNX₂NX₃LA |
| | 1*01 (B3) | WYQQKPGQPP KLLIYX₄ASTR ESGVPDRFSG SGSGTDFTLT |
| | | ISSLQAEDVA VYYC |
| | | Where X₁ may be A, F, H, S, or Y; X₂ may be E, |
| | | K, N, or T; X₃ may be A, D, F, H, N, S, W, or |
| | | Y; X₄ may be A, D, S, W or Y. |
| 9 | L-CDR3 | QQX₁X₂X₃X₄PX₅T |
| | formula | Where X₁ may be A, D, F, H, P, S, or Y; X₂ may |
| | | be D, E, G, H, I, K, N, R, T or Y; X₃ may be |
| | | D, G, H, K, N, S, T, or R; X₄ may be F, I, L, |
| | | N, R, W or Y as defined in Table 2. |
| 10 | JK4 | FGQGTKVEIK |
| 11 | human ATX | MARRSSFQSCQIISLFTFAVGVNICLGFTAHRIKRAEGWEEGPPTV |
| | isoform beta, | LSDSPWTNISGSCKGRCFELQEAGPPDCRCDNLCKSYTSCCHDFDE |
| | 863 aa | LCLKTARGWECTKDRCGEVRNEENACHCSEDCLARGDCCTNYQVVC |
| | | KGESHWVDDDCEEIKAAECPAGFVRPPLIIFSVDGFRASYMKKGSK |
| | | VMPNIEKLRSCGTHSPYMRPVYPTKTFPNLYTLATGLYPESHGIVG |
| | | NSMYDPVFDATFHLRGREKFNHRWWGGQPLWITATKQGVKAGTFFW |
| | | SVVIPHERRILTILQWLTLPDHERPSVYAFYSEQPDFSGHKYGPFG |
| | | PEMTNPLREIDKIVGQLMDGLKQLKLHRCVNVIFVGDHGMEDVTCD |
| | | RTEFLSNYLTNVDDITLVPGTLGRIRSKFSNNAKYDPKAIIANLTC |
| | | KKPDQHFKPYLKQHLPKRLHYANNRRIEDIHLLVERRWHVARKPLD |
| | | VYKKPSGKCFFQGDHGFDNKVNSMQTVFVGYGSTFKYKTKVPPFEN |
| | | IELYNVMCDLLGLKPAPNNGTHGSLNHLLRTNTFRPTMPEEVTRPN |
| | | YPGIMYLQSDFDLGCTCDDKVEPKNKLDELNKRLHTKGSTEERHLL |
| | | YGRPAVLYRTRYDILYHTDFESGYSEIFLMPLWTSYTVSKQAEVSS |
| | | VPDHLTSCVRPDVRVSPSFSQNCLAYKNDKQMSYGFLFPPYLSSSP |
| | | EAKYDAFLVTNMVPMYPAFKRVWNYFQRVLVKKYASERNGVNVISG |
| | | PIFDYDYDGLHDTEDKIKQYVEGSSIPVPTHYYSIITSCLDFTQPA |
| | | DKCDGPLSVSSFILPHRPDNEESCNSSEDESKWVEELMKMHTARVR |
| | | DIEHLTSLDFFRKTSRSYPEILTLKTYLHTYESEI |
| 12 | human ATX | MARRSSFQSCQIISLFTFAVGVNICLGFTAHRIKRAEGWEEGPPTV |
| | isoform alpha, | LSDSPWTNISGSCKGRCFELQEAGPPDCRCDNLCKSYTSCCHDFDE |
| | 915 aa | LCLKTARGWECTKDRCGEVRNEENACHCSEDCLARGDCCTNYQVVC |
| | | KGESHWVDDDCEEIKAAECPAGFVRPPLIIFSVDGFRASYMKKGSK |
| | | VMPNIEKLRSCGTHSPYMRPVYPTKTFPNLYTLATGLYPESHGIVG |
| | | NSMYDPVFDATFHLRGREKFNHRWWGGQPLWITATKQGVKAGTFFW |
| | | SVVIPHERRILTILQWLTLPDHERPSVYAFYSEQPDFSGHKYGPFG |
| | | PEESSYGSPFTPAKRPKRKVAPKRRQERPVAPPKKRRRKIHRMDHY |
| | | AAETRQDKMTNPLREIDKIVGQLMDGLKQLKLHRCVNVIFVGDHGM |
| | | EDVTCDRTEFLSNYLTNVDDITLVPGTLGRIRSKFSNNAKYDPKAI |
| | | IANLTCKKPDQHFKPYLKQHLPKRLHYANNRRIEDIHLLVERRWHV |
| | | ARKPLDVYKKPSGKCFFQGDHGFDNKVNSMQTVFVGYGSTFKYKTK |
| | | VPPFENIELYNVMCDLLGLKPAPNNGTHGSLNHLLRTNTFRPTMPE |
| | | EVTRPNYPGIMYLQSDFDLGCTCDDKVEPKNKLDELNKRLHTKGST |
| | | EERHLLYGRPAVLYRTRYDILYHTDFESGYSEIFLMPLWTSYTVSK |
| | | QAEVSSVPDHLTSCVRPDVRVSPSFSQNCLAYKNDKQMSYGFLFPP |
| | | YLSSSPEAKYDAFLVTNMVPMYPAFKRVWNYFQRVLVKKYASERNG |
| | | VNVISGPIFDYDYDGLHDTEDKIKQYVEGSSIPVPTHYYSIITSCL |
| | | DFTQPADKCDGPLSVSSFILPHRPDNEESCNSSEDESKWVEELMKM |
| | | HTARVRDIEHLTSLDFFRKTSRSYPEILTLKTYLHTYESEI |
| 13 | human ATX | MARRSSFQSCQIISLFTFAVGVNICLGFTAHRIKRAEGWEEGPPTV |
| | isoform | LSDSPWTNISGSCKGRCFELQEAGPPDCRCDNLCKSYTSCCHDFDE |
| | gamma, 888 aa | LCLKTARGWECTKDRCGEVRNEENACHCSEDCLARGDCCTNYQVVC |
| | | KGESHWVDDDCEEIKAAECPAGFVRPPLIIFSVDGFRASYMKKGSK |
| | | VMPNIEKLRSCGTHSPYMRPVYPTKTFPNLYTLATGLYPESHGIVG |
| | | NSMYDPVFDATFHLRGREKFNHRWWGGQPLWITATKQGVKAGTFFW |
| | | SVVIPHERRILTILQWLTLPDHERPSVYAFYSEQPDFSGHKYGPFG |
| | | PEMTNPLREIDKIVGQLMDGLKQLKLHRCVNVIFVGDHGMEDVTCD |
| | | RTEFLSNYLTNVDDITLVPGTLGRIRSKFSNNAKYDPKAIIANLTC |
| | | KKPDQHFKPYLKQHLPKRLHYANNRRIEDIHLLVERRWHVARKPLD |
| | | VYKKPSGKCFFQGDHGFDNKVNSMQTVFVGYGSTFKYKTKVPPFEN |
| | | IELYNVMCDLLGLKPAPNNGTHGSLNHLLRTNTFRPTMPEEVTRPN |
| | | YPGIMYLQSDFDLGCTCDDKVEPKNKLDELNKRLHTKGSTEAETRK |
| | | X1RGX2RNENKENINGNFEPRKERHLLYGRPAVLYRTRYDILYHTD |
| | | FESGYSEIFLMPLWTSYTVSKQAEVSSVPDHLTSCVRPDVRVSPSF |
| | | SQNCLAYKNDKQMSYGFLFPPYLSSSPEAKYDAFLVTNMVPMYPAF |
| | | KRVWNYFQRVLVKKYASERNGVNVISGPIFDYDYDGLHDTEDKIKQ |
| | | YVEGSSIPVPTHYYSIITSCLDFTQPADKCDGPLSVSSFILPHRPD |
| | | NEESCNSSEDESKWVEELMKMHTARVRDIEHLTSLDFFRKTSRSYP |
| | | EILTLKTYLHTYESEI |
| 14 | Mus spps. ATX | MARQGCFGSYQVISLFTFAIGVNLCLGFTASRIKRAEWDEGPPTVL |
| | | SDSPWTNTSGSCKGRCFELQEVGPPDCRCDNLCKSYSSCCHDFDEL |
| | | CLKTARGWECTKDRCGEVRNEENACHCSEDCLSRGDCCTNYQVVCK |
| | | GESHWVDDDCEEIRVPECPAGFVRPPLIIFSVDGFRASYMKKGSKV |
| | | MPNIEKLRSCGTHAPYMRPVYPTKTFPNLYTLATGLYPESHGIVGN |
| | | SMYDPVFDATFHLRGREKFNHRWWGGQPLWITATKQGVRAGTFFWS |
| | | VSIPHERRILTILQWLSLPDNERPSVYAFYSEQPDFSGHKYGPFGP |
| | | EMTNPLREIDKTVGQLMDGLKQLKLHRCVNVIFVGDHGMEDVTCDR |
| | | TEFLSNYLTNVDDITLVPGTLGRIRPKIPNNLKYDPKAIIANLTCK |
| | | KPDQHFKPYMKQHLPKRLHYANNRRIEDLHLLVERRWHVARKPLDV |
| | | YKKPSGKCFFQGDHGFDNKVNSMQTVFVGYGPTFKYRTKVPPFENI |
| | | ELYNVMCDLLGLKPAPNNGTHGSLNHLLRTNTFRPTLPEEVSRPNY |
| | | PGIMYLQSDFDLGCTCDDKVEPKNKLEELNKRLHTKGSTEERHLLY |
| | | GRPAVLYRTSYDILYHTDFESGYSEIFLMPLWTSYTISKQAEVSSI |
| | | PEHLTNCVRPDVRVSPGFSQNCLAYKNDKQMSYGFLFPPYLSSSPE |
| | | AKYDAFLVTNMVPMYPAFKRVWTYFQRVLVKKYASERNGVNVISGP |
| | | IFDYNYNGLRDIEDEIKQYVEGSSIPVPTHYYSIITSCLDFTQPAD |
| | | KCDGPLSVSSFILPHRPDNDESCNSSEDESKWVEELMKMHTARVRD |
| | | IEHLTGLDFYRKTSRSYSEILTLKTYLHTYESEI |
| 15 | Purification | GSHHHHHH |
| | Tag | fused at the N-terminal of position 49 of ATX |
| | | proteins |
| 16 | H4 in B1 | EVQLVQSGAEVKKPGESLKISCKGSGYSFTSYWIGWVRQMPGKGLE |
| | | WMGIIYPGDSDTRYSPSFQGQVTISADKSISTAYLQWSSLKASDTA |
| | | MYYCARNWSEALYVVFDY |
| 17 | L4 in B1 | EIVLTQSPATLSLSPGERATLSCRASQSVANFLAWYQQKPGQAPRL |
| | | LIYYASNRATGIPARFSGSGSGTDFTLTISSLEPEDFAVYYCQQYF |
| | | SAPFT |
| 18 | H10 in B2 | EVQLVQSGAEVKKPGESLKISCKGSGYSFTSYWIGWVRQMPGKGLE |
| | | WMGIIYPGDSDTRYSPSFQGQVTISADKSISTAYLQWSSLKASDTA |
| | | MYYCARGLADFWYFDY |
| 19 | L13 in B2 | DIQMTQSPSSLSASVGDRVTITCRASQSIDVWLNWYQQKPGKAPKL |
| | | LIYAASSLQSGVPSRFSGSGSGTDFTLTISSLQPEDFATYYCQQSY |
| | | STPLT |
| 20 | H11 in B3 | EVQLVQSGAEVKKPGESLKISCKGSGYSFTSYWIGWVRQMPGKGLE |
| | | WMGIIYPGDSDTRYSPSFQGQVTISADKSISTAYLQWSSLKASDTA |
| | | MYYCARPYGTFDY |
| 21 | L14 in B3 | EIVLTQSPATLSLSPGERATLSCRASQSVADSLAWYQQKPGQAPRL |
| | | LIYKASNRATGIPARFSGSGSGTDFTLTISSLEPEDFAVYYCQQSN |
| | | GWPRT |
| 22 | H2 in B4 and | QVQLVQSGAEVKKPGSSVKVSCKASGGTFSSYAISWVRQAPGQGLE |
| | B5 | WMGGIIPIFGTANYAQKFQGRVTITADESTSTAYMELSSLRSEDTA |
| | | VYYCARDDLPLGRLDY |
| 23 | L30 in B4 | EIVLTQSPATLSLSPGERATLSCRASQSVNKHLAWYQQKPGQAPRL |
| | | LIYKASNRATGIPARFSGSGSGTDFTLTISSLEPEDFAVYYCQQGI |
| | | RAPYT |
| 24 | H66 in B6 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARDLYIYGDLDY |
| 25 | L28 in B5 | EIVLTQSPATLSLSPGERATLSCRASQSVAKFLAWYQQKPGQAPRL |
| | | LIYKASNRATGIPARFSGSGSGTDFTLTISSLEPEDFAVYYCQQFN |
| | | SAPITF |
| 26 | H80 in B7 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARPIGTGYYGFFDY |
| 27 | L67 in B6 | DIQMTQSPSSLSASVGDRVTITCRASQSIGGWLNWYQQKPGKAPKL |
| | | LIYTASSLQSGVPSRFSGSGSGTDFTLTISSLQPEDFATYYCQQSY |
| | | TYPIT |
| 28 | H89 in B8 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARDSSAYGDFDY |
| 29 | L80 in B7 | DIQMTQSPSSLSASVGDRVTITCRASQSIAKWLNWYQQKPGKAPKL |
| | | LIYFASSLQSGVPSRFSGSGSGTDFTLTISSLQPEDFATYYCQQSY |
| | | STPLT |
| 30 | H70 in B9 | QVQLVQSGAEVKKPGSSVKVSCKASGGTFSSYAISWVRQAPGQGLE |
| | | WMGGISPYFGNANYAQKFQGRVTITADESTSTAYMELSSLRSEDTA |
| | | VYYCARYGDFGGNVFTEFDY |
| 31 | VL-IFWL124 in | DIQMTQSPSSLSASVGDRVTITCRASQSIDGWLNWYQQKPGKAPKL |
| | B8, B14, and | LIYAASSLQSGVPSRFSGSGSGTDFTLTISSLQPEDFATYYCQQSY |
| | B18 | STPLT |
| 32 | H60 in B10 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARIARYARGYMHTFDY |
| 33 | L82 in B9 | EIVLTQSPGTLSLSPGERATLSCRASQSVNNSDLAWYQQKPGQAPR |
| | | LLIYTASSRATGIPDRFSGSGSGTDFTLTISRLEPEDFAVYYCQQA |
| | | TTVPLT |
| 34 | H111 in B11 | QVQLVQSGAEVKKPGSSVKVSCKASGGTFSSYGISWVRQAPGQGLE |
| | | WMGGIIPIFGTANYAQKFQGRVTITADESTSTAYMELSSLRSEDTA |
| | | VYYCARDDLPLGMLDY |
| 35 | L61 in B10 | DIQMTQSPSSLSASVGDRVTITCRASQSISSYLNWYQQKPGKAPKL |
| | | LIYAASSLQSGVPSRFSGSGSGTDFTLTISSLQPEDFATYYCQQHL |
| | | SIPIT |
| 36 | H123 in B12 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARDRYYFTVLDY |
| 37 | L104 in B11 | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQY |
| | | TAFPLT |
| 38 | H148 in B13 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARDFSVYGDFDY |
| 39 | L110 in B12 | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQA |
| | | TAFPLT |
| 40 | H96 in B14 | QVQLVQSGAEVKKPGSSVKVSCKASGGTFSSYAISWVRQAPGQGLE |
| | | WMGGIIPIFGNTNYAQKFQGRVTITADESTSTAYMELSSLRSEDTA |
| | | VYYCARDISGYGDLDY |
| 41 | H2RL12 in B13 | DIQMTQSPSSLSASVGDRVTITCRASQSIAKWLNWYQQKPGKAPKL |
| | | LIYAASSLQSGVPSRFSGSGSGTDFTLTISSLQPEDFATYYCQQSY |
| | | STPLT |
| 42 | H153 in B15 | EVQLVQSGGGLVQPGGSLRLSCAASGFTFSSYAMSWVRQAPGKGLE |
| | | WVSAISGSGGSTYYADSVKGRFTISRDNSKNTLYLQMNSLRAEDTA |
| | | VYYCAKNAWEGAMLDY |
| 43 | L35 in B15 | DIQMTQSPSSLSASVGDRVTITCRASQSIHNWLNWYQQKPGKAPKL |
| | | LIYAASSLQSGVPSRFSGSGSGTDFTLTISSLQPEDFATYYCQQSY |
| | | STPLT |
| 44 | H154 in B16 | EVQLVQSGGGLVQPGGSLRLSCAASGFTFSSYAMSWVRQAPGKGLE |
| | | WVSVINSDGSSIYYADSVKGRFTISRDNSKNTLYLQMNSLRAEDTA |
| | | VYYCAKLEAIDYAWGFDY |
| 45 | L42 in B16 | EIVLTQSPATLSLSPGERATLSCRASQSVADSLAWYQQKPGQAPRL |
| | | LIYNASNRATGIPARFSGSGSGTDFTLTISSLEPEDFAVYYCQQSF |
| | | HWPLT |
| 46 | H155 in B17 | EVQLVQSGGGLVQPGGSLRLSCAASGFTFSSYAMSWVRQAPGKGLE |
| | | WVSAISGSGGSTYYADSVKGRFTISRDNSKNTLYLQMNSLRAEDTA |
| | | VYYCAKDVKGSATLDY |
| 47 | IFWL195 in | DIQMTQSPSSLSASVGDRVTITCRASQSIANWLNWYQQKPGKAPKL |
| | B17 | LIYAASSLQSGVPSRFSGSGSGTDFTLTISSLQPEDFATYYCQQSY |
| | | STPLT |
| 48 | H90 in B18 | QVQLVQSGAEVKKPGSSVKVSCKASGGTFSSYAISWVRQAPGQGLE |
| | | WMGGIIPIFGTANYAQKFQGRVTITADESTSTAYMELSSLRSEDTA |
| | | VYYCARDASTYGDLDY |
| 49 | CDR-H1 | SYAIS |
| 50 | CDR-H1 | SYGIS |
| 51 | CDR-H1 | SYAMS |
| 52 | CDR-H1 | SYWIG |
| 53 | CDR-H2 | GIIPIFGTANYAQKFQG |
| 54 | CDR-H2 | GIIPIFGNTNYAQKFQG |
| 55 | CDR-H2 | GISPYFGTANYAQKFQG |
| 56 | CDR-H2 | AISGSGGSTYYADSVKG |
| 57 | CDR-H2 | VINSDGSSIYYADSVKG |
| 58 | CDR-H2 | IIYPGDSDTRYSPSFQG |
| 59 | CDR-H2 | GIIPIFGNANYAQKFQG |
| 60 | CDR-H2 | GIIPIFGTTNYAQKFQG |
| 61 | CDR-H3 | DLYIYGDLDY |
| 62 | CDR-H3 | DSSAYGDFDY |
| 63 | CDR-H3 | DASTYGDLDY |
| 64 | CDR-H3 | DISGYGDLDY |
| 65 | CDR-H3 | DFSVYGDFDY |
| 66 | CDR-H3 | PIGTGYYGFFDY |
| 67 | CDR-H3 | YGDFGGNVFTEFDY |
| 68 | CDR-H3 | IARYARGYMHTFDY |
| 69 | CDR-H3 | DDLPLGRLDY |
| 70 | CDR-H3 | DDLPLGMLDY |
| 71 | CDR-H3 | DRYYFTVLDY |
| 72 | CDR-H3 | KNAWEGAMLDY |
| 73 | CDR-H3 | KDVKGSATLDY |
| 74 | CDR-H3 | KLEAIDYAWGFDY |
| 75 | CDR-H3 | GLADFWYFDY |
| 76 | CDR-H3 | PYGTFDY |
| 77 | CDR-H3 | NWSEALYVVFDY |
| 78 | CDR-H3 | DDVALGRLDY |
| 79 | CDR-H3 | SDLNNVSTVLDY |
| 80 | CDR-H3 | ADANQLSGYLDY |
| 81 | CDR-H3 | EDVALGRLDY |
| 82 | CDR-H3 | HDVNSLWAYFDY |
| 83 | CDR-H3 | GDYNAIYYALDY |
| 84 | CDR-H3 | DDLALGRLDY |
| 85 | CDR-H3 | DDDVNSLASYPLDY |
| 86 | CDR-H3 | DGDVNSVYSSGLDY |
| 87 | CDR-H3 | GDANALYDYFDY |
| 88 | CDR-H3 | DDWLSHFDY |
| 89 | CDR-H3 | RDGNYLHVDHFDY |
| 90 | CDR-H3 | DHGYAYYSAFDY |
| 91 | CDR-H3 | DFLSQFSWLDY |
| 92 | CDR-H3 | DGVTTYDYFDY |
| 93 | CDR-H3 | HYLPDATLDY |
| 94 | CDR-H3 | GTFTYDFLDY |
| 95 | CDR-H3 | GFVNFSRLDY |
| 96 | CDR-H3 | DWWVTDILDY |
| 97 | CDR-H3 | GEANEVYPGLDY |
| 98 | CDR-H3 | DLYGIDIYYALDY |
| 99 | CDR-H3 | DTFWFTVFDY |
| 100 | CDR-H3 | GDANSIYPLFDY |
| 101 | CDR-H3 | GADSNAVRLVGFDY |
| 102 | CDR-H3 | EDVNYISTYSEFDY |
| 103 | CDR-H3 | GDDNEVIYHLDY |
| 104 | CDR-L1 | RASQSIGGWLN |
| 105 | CDR-L1 | RASQSIDGWLN |
| 106 | CDR-L1 | RASQSIAKWLN |
| 107 | CDR-L1 | RASQSVNNSDLA |
| 108 | CDR-L1 | RASQSISSYLN |
| 109 | CDR-L1 | RASQSVNKHLA |
| 110 | CDR-L1 | RASQSVAKFLA |
| 111 | CDR-L1 | RASQSVSSSYLA |
| 112 | CDR-L1 | RASQSIHNWLN |
| 113 | CDR-L1 | RASQSIANWLN |
| 114 | CDR-L1 | RASQSVADSLA |
| 115 | CDR-L1 | RASQSIDVWLN |
| 116 | CDR-L1 | RASQSVANFLA |
| 117 | CDR-L1 | RASQSIGKVLN |
| 118 | CDR-L1 | RASQSIGRSLN |
| 119 | CDR-L1 | RASQSVSKALA |
| 120 | CDR-L1 | RASQSVSSFLA |
| 121 | CDR-L1 | RASQSVNSWLA |
| 122 | CDR-L1 | RASQSVADFLA |
| 123 | CDR-L1 | RASQSVSDYLA |
| 124 | CDR-L1 | RASQSVNKFLA |
| 125 | CDR-L1 | RASQSVRDWLA |
| 126 | CDR-L1 | RASQSVRKYLA |
| 127 | CDR-L1 | RASQSVRNFLA |
| 128 | CDR-L1 | RASQSVSNWLA |
| 129 | CDR-L1 | RASQSVNNFLA |
| 130 | CDR-L1 | RASQSVADWLA |
| 131 | CDR-L1 | RASQSVSSWLA |
| 132 | CDR-L1 | RASQSVADYLA |
| 133 | CDR-L1 | RASQSVAKDLA |
| 134 | CDR-L1 | RASQSVASALA |
| 135 | CDR-L1 | RASQSVSKSALA |
| 136 | CDR-L1 | RASQSVAYSSLA |
| 137 | CDR-L1 | RASQSVRKSGLA |
| 138 | CDR-L1 | RASQSVAKSDLA |
| 139 | CDR-L2 | TASSLQS |
| 140 | CDR-L2 | AASSLQS |
| 141 | CDR-L2 | FASSLQS |
| 142 | CDR-L2 | TASSRAT |
| 143 | CDR-L2 | KASNRAT |
| 144 | CDR-L2 | GASSRAT |
| 145 | CDR-L2 | NASNRAT |
| 146 | CDR-L2 | YASNRAT |
| 147 | CDR-L2 | GASSLQS |
| 148 | CDR-L2 | KASSLQS |
| 149 | CDR-L2 | TASNRAT |
| 150 | CDR-L2 | DASNRAT |
| 151 | CDR-L2 | FASNRAT |
| 152 | CDR-L2 | GASHRAT |
| 153 | CDR-L2 | NASSRAT |
| 154 | CDR-L2 | AASSRAT |
| 155 | CDR-L3 | QQSYTYPIT |
| 156 | CDR-L3 | QQSYSTPLT |
| 157 | CDR-L3 | QQATTVPLT |
| 158 | CDR-L3 | QQHLSIPIT |
| 159 | CDR-L3 | QQGIRAPYT |
| 160 | CDR-L3 | QQFNSAPIT |
| 161 | CDR-L3 | QQYTAFPLT |
| 162 | CDR-L3 | QQATAFPLT |
| 163 | CDR-L3 | QQSFHNPLT |
| 164 | CDR-L3 | QQSHGWPRT |
| 165 | CDR-L3 | QQYFSAPFT |
| 166 | CDR-L3 | QQPISTPLT |
| 167 | CDR-L3 | QQPFDYPFT |
| 168 | CDR-L3 | QQPISFPYT |
| 169 | CDR-L3 | QQGHGWPPT |
| 170 | CDR-L3 | QQYKMAPRT |
| 171 | CDR-L3 | QQSHRAPWT |
| 172 | CDR-L3 | QQGHPWPFT |
| 173 | CDR-L3 | QQGSTAPYT |
| 174 | CDR-L3 | QQGSDAPLT |
| 175 | CDR-L3 | QQYHHAPFT |
| 176 | CDR-L3 | QQGDGWPWT |
| 177 | CDR-L3 | QQGDTAPI |
| 178 | CDR-L3 | QQGYRAPIT |
| 179 | CDR-L3 | QQRYTAPWT |
| 180 | CDR-L3 | QQFKGAPLT |
| 181 | CDR-L3 | QQSFRWPLT |
| 182 | CDR-L3 | QQFKGWPFT |
| 183 | CDR-L3 | QQYIRWPIT |
| 184 | CDR-L3 | QQYISAPLT |
| 185 | CDR-L3 | QQGINWPFT |
| 186 | CDR-L3 | QQGGNWPWT |
| 187 | CDR-L3 | QQYSTWPFT |
| 188 | CDR-L3 | QQSSRDPWT |
| 189 | CDR-L3 | QQSSGFPLT |
| 190 | CDR-L3 | QQSSRYPWT |
| 191 | CDR-L3 | QQYGSSPLT |
| 192 | CDR-L3 | QQSSRFPWT |
| 193 | CDR-L3 | QQDSAFPWT |
| 194 | CDR-L3 | QQSNTNPLT |
| 195 | CDR-L3 | QQSNKDPLT |
| 196 | CDR-L3 | QQSNTLPLT |
| 197 | CDR-L3 | QQSNTPPLT |
| 198 | CDR-L3 | QQATKPPLT |
| 199 | CDR-L3 | QQDYGYPLT |
| 200 | consensus | QVQLVQSGAEVKKPGSSVKVSCKASGGTFSSYAISWVRQAPGQGLE |
| | sequence for | WMGGI X1P X2FG |
| | an ATX | X3X4NYAQKFQGRVTITADESTSTAYMELSSLRSEDTAVYYCARDL |
| | neutralizing | YIYG X5 X6DY |
| | human IGHV1- | wherein X1 is I or S, X2 is I or Y, X3 is N or T, |
| | 69 germline | X4 is A or T, X5 is D or F and X5 is F or L |
| | derived VH | |
| 201 | consensus | |
| | sequence for | DIQMTQSPSSLSASVGDRVTITCRASQSI |
| | an ATX | X₁X₂WLNWYQQKPGKAPKLLIYX₃ASSLQSGVPSRFSGSGSGTDFTL |
| | neutralizing | TISSLQPEDFATYYCQQSY X₄ X₅P X₆T |
| | human gene | wherein X₁ is A, D or G; X₂ is G or K; and X₃ is |
| | derived | A or T; and X₄ is S or T; X₅ is T or Y; and X₆ |
| | IGKV1-39 | is I or L |
| | (O12) VL | |
| 202 | Homo sapiens | QVQLVQSGAEVKKPGSSVKVSCKASGGTFSSYAISWVRQAPGQGLE |
| | IGHV1-69 | WMGGIIPIFGTANYAQKFQGRVTITADESTSTAYMELSSLRSEDTA |
| | germline gene | VYYCARWGQGTLVTVSS |
| | with joining | |
| | JH | |
| 203 | Homo sapiens | EVQLLESGGGLVQPGGSLRLSCAASGFTFSSYAMSWVRQAPGKGLE |
| | IGHV3-23 | WVSAISGSGGSTYYADSVKGRFTISRDNSKNTLYLQMNSLRAEDTA |
| | germline gene | VYYCAKWGQGTLVTVSS |
| | with joining | |
| | JH | |
| 204 | Homo sapiens | EVQLVQSGAEVKKPGESLKISCKGSGYSFTSYWIGWVRQMPGKGLE |
| | IGHV5-51 | WMGIIYPGDSDTRYSPSFQGQVTISADKSISTAYLQWSSLKASDTA |
| | germline gene | MYYCARWGQGTLVTVSS |
| | with joining | |
| | JH | |
| 205 | IGKV3-20 | EIVLTQSPGTLSLSPGERATLSCRASQSVSSSYLAWYQQKPGQAPR |
| | germline gene | LLIYGASSRATGIPDRFSGSGSGTDFTLTISRLEPEDFAVYYCQQY |
| | with joining | GSSPLTFGQGTKVEIK |
| | JK | |
| 206 | Homo sapiens | DIVMTQSPDSLAVSLGERATINCKSSQSVLYSSNNKNYLAWYQQKP |
| | IGKV4-1 | GQPPKLLIYWASTRESGVPDRFSGSGSGTDFTLTISSLQAEDVAVY |
| | germline gene | YCQQYYSTPLTFGQGTKVEIK |
| | with joining | |
| | JK | |
| 207 | Homo sapiens | EIVLTQSPATLSLSPGERATLSCRASQSVSSYLAWYQQKPGQAPRL |
| | IGKV3-11 | LIYDASNRATGIPARFSGSGSGTDFTLTISSLEPEDFAVYYCQQRS |
| | germline gene | NWPLTFGQGTKVEIK |
| | with joining | |
| | JK | |
| 208 | Homo sapiens | DIQMTQSPSSLSASVGDRVTITCRASQSISSYLNWYQQKPGKAPKL |
| | IGKV1-39 | LIYAASSLQSGVPSRFSGSGSGTDFTLTISSLQPEDFATYYCQQSY |
| | germline gene | STPLTFGQGTKVEIK |
| | with joining | |
| | JK | |

What is claimed:

1. An isolated monoclonal antibody that specifically binds to isolated, catalytically active, human autotaxin (ATX) protein with a K_{D} of less than 5×10⁻⁸ M as measured by surface plasmon resonance (SPR).

2. The antibody of claim 1, which reduces lysophospholipase D activity of ATX as measured by the conversion of lysophosphatidylcholine to lysophosphatidic acid and choline.

3. The antibody of claim 2 comprising
a. a heavy chain complementarity determining region (HCDR) 1 (HCDR1) of amino acid residues 35-49 of SEQ ID NO: 200;
b. a HCDR2 of amino acid residues 64-80 of SEQ ID NO: 200;
c. a HCDR3 of amino acid residues 113-122 of SEQ ID NO: 200;
d. a light chain complementarity determining-region (LCDR) 1 (LCDR1) of amino acid residues 24-34 of SEQ ID NO: 201;
e. a LCDR2 of amino acid residues 50-56 of SEQ ID NO: 201; and
f. a LCDR3 of amino acid residues 89-97 of SEQ ID NO: 201.

4. The antibody of claim 3 further comprising the HCDR1 sequences of SEQ ID NO: 49, the HCDR2 sequences of the sequences selected from the group consisting of SEQ ID NOs: 53 and 54, the HCDR3 sequences selected from the group consisting of SEQ ID NOs: 61-65 and 66, the LCDR1 sequences selected from the group consisting of SEQ ID NOs: 104, 105 and 106, the LCDR2 sequences of the sequences selected from the group consisting of SEQ ID NOs: 139, 140 and 141, and the LCDR3 sequences selected from the group consisting of SEQ ID NOs: 155 and 156.

5. The antibody of claim 3 comprising a variable heavy (VH) and a variable light (VL) domain, wherein the VH is derived from IGHV1-69 (SEQ ID NO: 202), IGHV3-23 (SEQ ID NO: 203) or IGHV5-51 (SEQ ID NO: 204) human germline genes and the VL is derived from IGKV1-39 (SEQ ID NO: 208), IGKV3-11 (SEQ ID NO: 207) or IGKV3-20 (SEQ ID NO: 205) human germline genes.

6. The antibody of claim 5, wherein the VH is derived from IGHV1-69 (SEQ ID NO: 202) human germline gene and the VL is derived from IGKV1-39 (SEQ ID NO: 208) or IGKV3-20 (SEQ ID NO: 205) human germline gene.

7. The isolated antibody of claim 6 comprising the VH of SEQ ID NO: 200 and the VL of SEQ ID NO: 201.

8. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 61;
d. the LCDR1 of SEQ ID NO:104;
e. the LCDR2 of SEQ Id NO: 139; and
f. the LCDR3 of SEQ ID NO: 155.

9. The antibody of claim 8, wherein the antibody comprises
a. the VH of SEQ ID NO: 24; and
b. the VL of SEQ ID NO: 27.

10. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 62;
d. the LCDR1 of SEQ ID NO:105;
e. the LCDR2 of SEQ Id NO: 140; and
f. the LCDR3 of SEQ ID NO: 156.

11. The antibody of claim 10, wherein the antibody comprises
a. the VH of SEQ ID NO: 24; and
b. the VL of SEQ ID NO: 31.

12. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 63;
d. the LCDR1 of SEQ ID NO:105;
e. the LCDR2 of SEQ Id NO: 140; and
f. the LCDR3 of SEQ ID NO: 156.

13. The antibody of claim 12, wherein the antibody comprises
a. the VH of SEQ ID NO: 48; and
b. the VL of SEQ ID NO: 31.

14. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 64;
d. the LCDR1 of SEQ ID NO:105;
e. the LCDR2 of SEQ Id NO: 140; and
f. the LCDR3 of SEQ ID NO: 156.

15. The antibody of claim 14, wherein the antibody comprises
a. the VH of SEQ ID NO: 40; and
b. the VL of SEQ ID NO: 31.

16. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 65;
d. the LCDR1 of SEQ ID NO:106;
e. the LCDR2 of SEQ Id NO: 140; and
f. the LCDR3 of SEQ ID NO: 156.

17. The antibody of claim 16, wherein the antibody comprises
a. the VH of SEQ ID NO: 38; and
b. the VL of SEQ ID NO: 41.

18. The isolated antibody of claim 6, wherein the antibody comprises
a. the HCDR1 of SEQ ID NO: 49;
b. the HCDR2 of SEQ ID NO: 53;
c. the HCDR3 of SEQ ID NO: 66;
d. the LCDR1 of SEQ ID NO:106;
e. the LCDR2 of SEQ Id NO: 141; and
f. the LCDR3 of SEQ ID NO: 156.

19. The antibody of claim 17, wherein the antibody comprises
a. the VH of SEQ ID NO: 26; and
b. the VL of SEQ ID NO: 29.

20. The isolated antibody of claim 3, wherein the antibody comprises
a. the HCDR1 of SEQ ID NO: 49;
b. the HCDR2 of SEQ ID NO: 55;
c. the HCDR3 of SEQ ID NO: 67;
d. the LCDR1 of SEQ ID NO:107;
e. the LCDR2 of SEQ Id NO: 142; and
f. the LCDR3 of SEQ ID NO: 157.

21. The antibody of claim 20, wherein the antibody comprises
a. the VH of SEQ ID NO: 30; and
b. the VL of SEQ ID NO: 33.

22. An isolated antibody that specifically binds to catalytically active human ATX according to claim 2, wherein the antibody is incapable of binding human ATX when an second antibody selected from the group consisting of B6, B8, B13, and B14 is pre-bound to the human ATX.

23. The isolated antibody of claim 22, wherein the antibody comprises a VH of SEQ ID NO: 200 and a VL of SEQ ID NO: 201.

24. The isolated antibody of claim 22, wherein an antibody paratope contacts epitope residues of human ATX having the amino acid sequence of SEQ ID NO: 11 at a residue selected from one or more of residues 201-255.

25. An isolated antibody that specifically binds to catalytically active human ATX according to claim 1, wherein the antibody is capable of binding human ATX when an second antibody selected from the group consisting of B6, B8, B13, and B14 is pre-bound to the human ATX.
